(19) United States
(12) Patent Application Publication
(10) Pub. No.: US 2011/0250222 A1
(43) Pub. Date: Oct. 13, 2011

(54) ANTIGENIC POLYPEPTIDES OF CHLAMYDIA-RELATED BACTERIA FOR DIAGNOSIS AND VACCINE

(75) Inventors: Gilbert Greub, Mollie-Margot (CH); Carole Kebbi Beghdadi, Lausanne (CH); Didier Raoult, Marseille (FR); Beat Riederer, Epalinges (CH)

(73) Assignee: UNIVERSITY OF LAUSANNE, Lausanne (CH)

(21) Appl. No.: 13/140,263

(22) PCT Filed: Dec. 4, 2009
(86) PCT No.: PCT/IB2009/055525

§ 371 Date: Jun. 16, 2011

(30) Foreign Application Priority Data
Dec. 18, 2008 (EP) 08172133.4

(51) Int. Cl. A61K 39/02; G01N 33/566; A61P 31/04; C07K 2/00; A61P 37/04; C07K 14/295; C07K 16/12

(52) U.S. Cl. 424/190.1; 530/350; 530/324; 436/501; 435/7.1; 530/387.9; 530/300

(57) ABSTRACT

The present invention relates to the disclosed transgenic peptides for use in the diagnosis of an infection by intracellular Chlamydia-like bacteria. The invention also relates to a serological diagnostic test. The transgenic peptides are selected from the proteome of Parachlamydia acanthamoebae properties of binding to antibodies of infected human and animals. The test may give further insight in the role of this microorganism in pulmonary diseases and possibly in miscarriage.

### TECHNICAL FIELD

The present invention generally relates to the fields of diagnosis, microbiology, immunology and more specifically to the use of recombinant and/or isolated peptides of the invention in the diagnosis of an infection by a Chlamydia-like microorganism, to a test for diagnosis, and to a method of diagnosis.

### PRIOR ART AND THE PROBLEM UNDERLYING THE INVENTION

Novel chlamydiae, amoebae-resisting bacteria, Chlamydia-like organisms, and Chlamydia-related bacteria are various acronyms used to refer to a large variety of strict intracellular bacteria belonging to the Chlamydiales order, but exhibiting enough biological differences with Chlamydiaceae to be assigned to other families. The biodiversity of these Chlamydia-related bacteria, as evidenced by both molecular-based studies and culture-based studies, led to the proposal of several new families, genus, species and Candidatus species.

Parachlamydia acanthamoebae strains have been first identified within Acanthamoeba amoebae isolated from the nasal mucosa of healthy volunteers. The role of these free-living amoebae as a widespread aquatic reservoir has thus been suspected early on.

By analogy with Legionella, P. acanthamoebae may have used amoebae as an evolutionary crib to select virulence traits, explaining its adaptation to survive within macrophages. Moreover, amoebae have likely played a pivotal role in the exchange of genes between Rickettsiales and Chlamydiales, as suggested by the occurrence of a similar tra operon in both clades.

The life cycle of P. acanthamoebae in Acanthamoeba has been thoroughly studied, demonstrating that Parachlamydia elementary bodies generally enter passively by phagocytosis before differentiating in reticulate bodies that may divide by binary fission, before re-differentiating in elementary bodies. Bacteria are then released from amoebae within expelled vesicles or after amoebal lysis. Crescent bodies of P. acanthamoebae, another infectious stage, may be observed (i) outside of the amoebae in the process of being internalized by phagocytosis or (ii) late in the intra-amoebal cycle, at a time when the amoeba is mainly filled with elementary bodies. These crescent bodies have also been observed among other Parachlamydiaceae, among Waddliaceae and to a lesser extend among Criblamydiaceae and Simkaniaceae. A peculiar composition of the cell membrane of these novel chlamydiae likely explains this special cell morphology.

The prevalence of human lung infections due to Parachlamydia is currently unknown. It may well be underestimated since these fastidious intracellular bacteria can only be cultivated from clinical samples using amoebal culture, a procedure not routinely performed in diagnostic laboratories. Human exposure to these Parachlamydiaceae is supported by the amplification of parachlamydial DNA from nose and/or throat swabs and recovery of two Parachlamydia strains from amoebae isolated from the nasal mucosa.

There are, however, several lines of evidence supporting the medical importance of P. acanthamoebae (Greub G, Raoult D. Parachlamydiaceae: potential emerging pathogens. Emerg Infect Dis 2002 June; 8(6):625-30). The first hint was the identification of P. acanthamoebae strain Hall's coccus within an amoeba isolated from the water of a humidifier linked to an outbreak of fever (Birtles R J, Rowbotham T J, Storey C, Marrie T J, Raoult D. Chlamydia-like obligate parasite of free-living amoebae. Lancet 1997 Mar. 29; 349(9056):925-6). This humidifier was only investigated for the presence of amoebae given the high attack rate observed and given the recognized role of amoebae in the epidemiology of Legionella, whose pathogenic role was also initially documented in a similar fashion during the original outbreak in 1976.

Several serological studies have suggested a role for P. acanthamoebae in community-acquired pneumonia. In one of these studies, 8 (2.2%) of 371 patients with community-acquired pneumonia exhibited a positive Parachlamydia serology as compared to 0 of 511 healthy controls (p<0.01) (Marrie T J, Raoult D, La Scola B, Birtles R J, de Carolis E. Legionella-like and other amoebal pathogens as agents of community-acquired pneumonia. Emerg Infect Dis 2001 November-December; 7(6):1026-9). In this study, 2 patients presented serological evidence of acute parachlamydial infection: a 68 year-old renal transplant recipient and a 21 year-old man with adult-onset Kawasaki disease. Greub G, Boyadjiev I, La Scola B, Raoult D, and Martin C. (Serological hint suggesting that Parachlamydiaceae are agents of pneumonia in polytraumatized intensive care patients. Ann N Y Acad Sci 2003 June; 990:311-9) also identified a possible role of Parachlamydia in aspiration pneumonia. Indeed, while investigating patients hospitalized on a neurosurgical ward for a possible association of P. acanthamoebae infection with nosocomial infection, we unexpectedly identified 3 seroconversions, which all occurred in patients with aspiration pneumonia.

The identification of 16SrRNA gene sequences of Parachlamydiaceae from bronchoalveolar lavage fluid and sputum are additional hints for a pathogenic role of P. acanthamoebae in lower respiratory tract infections. Parachlamydial DNA was also amplified from monocytes taken from a patient with bronchitis (Ossewaarde J M, Meijer A. Molecular evidence for the existence of additional members of the order Chlamydiales. Microbiology 1999 February; 145 (Pt 2):411-7). Finally, P. acanthamoebae was amplified from nasopharyngeal swabs taken from children suffering from bronchiolitis of unexplained etiology (Casson N, Posfay-Barbe K M, Gervaix A, Greub G. A new diagnostic real-time PCR for the specific detection of P. acanthamoebae DNA in clinical samples. J Clin Microbiol 2008 Jan. 30).

In summary, a possible role is emerging for Parachlamydia as agent of bronchitis, bronchiolitis, community-acquired pneumonia and aspiration pneumonia.

When further investigating the pathogenic role of P. acanthamoebae, its ability to enter within human macrophages was found. This strict intracellular bacterium is able to enter and replicate within human monocyte-derived macrophages (Greub G, Mege J L, Raoult D. P. acanthamoebae enters and multiplies within human macrophages and induces their apoptosis Infect Immun 2003 October; 71(10):5979-85), representing a good example of the adaptation of an amoebae-resisting Chlamydiae to macrophages. However, contrary to Chlamydiaceae that mainly survive within the target cells by relocalizing in vacuoles associated with the exocytic pathway, P. acanthamoebae was able to survive within endocytic acidic vacuoles, by preventing the acquisition of cathepsin, a lysosomal hydrolase (Greub G, Mege J L, Gorvel J P, Raoult D, Meresse S. Intracellular trafficking of Parachlamydia acanthamoebae. Cell Microbiol 2005 April; 7(4):581-9). Moreover, P. acanthamoebae did not induce significant cytokine production and remain partially unrecognized by human macrophages (Greub G, Desnues B, Raoult D, Mege J L. Lack of microbicidal response in human macrophages infected with Parachlamydia acanthamoebae. Microbes Infect 2005 April; 7(4):714-9). However, parachlamydial macrophage infection was only partially successful since this strict intracellular bacteria only replicated to a low extent and induced the apoptosis of the macrophage (Greub et. al, Infect Immun 2003), thereby preventing its use as a replicative niche.

The permissiveness of lung fibroblasts and pneumocytes, and the absence of cytopathic effect on these cells were consequently of importance, since these lung cells may allow sustained parachlamydial viability for prolonged periods (Casson N, Medico N, Bille J, Greub G. Parachlamydia acanthamoebae enters and multiplies within pneumocytes and lung fibroblasts. Microbes Infect 2006 April; 8(5):1294-300).

Based on these in vitro studies, further studies were conducted that demonstrate that P. acanthamoebae may cause a severe pneumonia in experimentally infected mice, thus fulfilling the 3^{rd} and 4^{th} Koch criteria for a pathogenic role of this intracellular bacterium.

In view of the above, it is an objective of the present invention to provide diagnostic tools to further precise the role of the obligatory intracellular bacteria of the genus Parachlamydia as human and/or animal pathogens.

It is a further objective to determine more accurately if Parachlamydia strains are agents of one or more lower respiratory tract diseases, and if they can cause one or more of bronchitis, bronchiolitis, community-acquired pneumonia and aspiration pneumonia.

It is a further objective of the present invention to provide a diagnostic tool for determining the presence or absence of an infection by a Parachlamydia strain. Preferably, such a diagnostic tool is specific to the genus Parachlamydia and/or even specific for P. acanthamoebae. Accordingly, the diagnostic tool is able to detect infection with Parachlamydia and to allow the conclusion that the infection is not due to another organism, in particular another Chlamydia-like organism outside the Parachlamydiacae family. There is an objective of avoiding false-positives in a method of diagnosis and/or a diagnostic tool.

It is a further objective of the invention to provide a diagnostic tool and/or a method of diagnosis that is sensitive and thus also detects low infection levels.

It is noted that the development of a diagnostic tool is complicated by the fact that it has so far not been possible to isolate parachlamydial strains from human samples, including samples from which parachlamydial DNA was amplified. This is likely due to the obligatory intracellular live of these strains, which is specific to species of amoeba and/or to certain types of cells. Parachlamydia does not or only slowly grow in most available model cells, such as eucaryotic cell lines Vero, McCoy and HeLa, or other cells, such as A549 pneumocytes and lung fibroblasts.

It is an objective of the present invention to provide a serological test for the determination of an infection by Parachlamydia. More specifically, it is an objective to provide an Enzyme-linked Immunosorbent Assay (ELISA) or a similar serological test.

It is a further objective of the present invention to provide an immunohistochemic assay, for example an epifluorescence immunoassay, and a method of detecting Parachlamydia or any Chlamydia-related bacteria in tissue samples.

It is also an objective of the present invention to provide antigen to which polyclonal and/or monoclonal antibodies may be raised and used to detect Parachlamydia and/or related bacteria in animal and/or humans tissues thanks to immunochemistry or similar approaches.

It is an objective of the present invention to provide immunogenic proteins that may be used in a vaccine.

### SUMMARY OF INVENTION

Remarkably, the present inventors identified immunogenic proteins of Chlamydia-like intracellular microorganisms. The proteins are useful in the diagnosis of an infection by Parachlamydia, advantageously by way of a serological test or using immunohistochemistry. For serological approaches, it is possible to rapidly test an individual for the presence of infection with Parachlamydia on the basis of a small blood sample, for example.

Accordingly, the present invention provides, in a first aspect, the at least one peptide for use in the diagnosis of an infection by a Chlamydia-like intracellular microorganism.

In a second aspect, the present invention provides at least one recombinant immunogenic peptide for the use in the diagnosis of an infection by an intracellular Chlamydia-like microorganism.

In a third aspect, the present invention provides least one isolated/purified immunogenic peptide for use in the diagnosis of an infection by an intracellular Chlamydia-like microorganism.

In a fourth aspect, the invention provides at least one peptide comprising: (a) amino acid sequences according to any one of SEQ. ID. NO.: 9, 2, 3, 6, 40, 1, 4, 5, 7, 8, 10-39, 41-57; (b) amino acid sequences having at least 80% of sequence identity with any one of the amino acid sequences mentioned under (a); and/or (c) fragments of an amino acid sequence mentioned under (a) and/or (b) above.

In a fourth aspect, the present invention provides a test, in particular a test kit, of diagnosis comprising one or more isolated, purified and/or recombinant immunogenic peptides of a Parachlamydiaceae microorganism, for example Parachlamydia acanthamoebae.

In a fifth aspect, the present invention provides a purified, recombinant immunogenic peptide for use in the diagnosis of an infection by a Parachlamydiaceae microorganism, wherein said peptide comprises:

(a) amino acid sequences according to any one of SEQ. ID. NO.: 9, 2, 3, 6, 40, 1, 4, 5, 7, 8, 10-39, 41-57;
(b) amino acid sequences having at least 80% of sequence identity with any one of the amino acid sequences mentioned under (a); and/or
(c) fragments comprising 10 or more successive amino acids of an amino acid sequence mentioned under (a) and/or (b) above.

In a sixth aspect, the present invention provides the peptides, variants and fragments as defined in the present specification in vaccines against infection of a Chlamydia-like microorganism.

In a seventh aspect, the present invention provides a method of producing an antibody, the method comprising the steps of exposing a mammal to at least one peptide comprising an amino acid sequence selected from:

(a) amino acid sequences according to any one of SEQ. ID. NO.: 9, 2, 3, 6, 40, 1, 4, 5, 7, 8, 10-39, 41-57;
(b) amino acid sequences having at least 80% of sequence identity with any one of the amino acid sequences mentioned under (a); and/or
(c) fragments comprising 10 or more successive amino acids of an amino acid sequence mentioned under (a) or (b) above; and, harvesting said antibody from serum of said mammal.

In a eight aspect, the present invention provides a method of diagnosing infection of an individual by intracellular Chlamydia-like organisms, the method comprising the steps of: contacting a blood or serum sample of an individual with one or more purified, recombinant, and immunogenic peptides of the Chlamydia-like organism, in particular as disclosed and defined in this specification; determining the presence or absence in said sample of an antibody specifically binding to at least one of said immunogenic peptides; and diagnosing an infection if an antibody is detected in the previous step.

In an aspect, the present invention provides antibodies as obtained, disclosed and/or defined in this specification.

In another aspect, the present invention provides a nucleotide sequence encoding a peptide, variant peptide or fragment peptide as described herein. For examples, the nucleotide sequence may be a DNA or an RNA sequence.

In an aspect, the present invention further relates to the of peptides disclosed herein for use in the diagnosis of diseases of the lungs and the respiratory tract (pulmonary diseases), such as bronchitis, bronchiolitis, and pneumonia, in particular community-acquired and/or aspiration pneumonia.

In further aspects, the present invention provides a pharmaceutical composition comprising at least one peptide as defined herein, as well as the peptides and compositions as disclosed and defined herein in therapeutic and/or prophylactic treatment of a disease.

In yet another aspect, the present invention further provides a test designed to assess a risk of miscarriage. In particular, in a further aspect, the present invention provides a test for identifying a risk of adverse pregnancy outcomes such as miscarriage.

Further aspects and preferred embodiment of the present invention are as provided in the appended claims.

In the drawings,

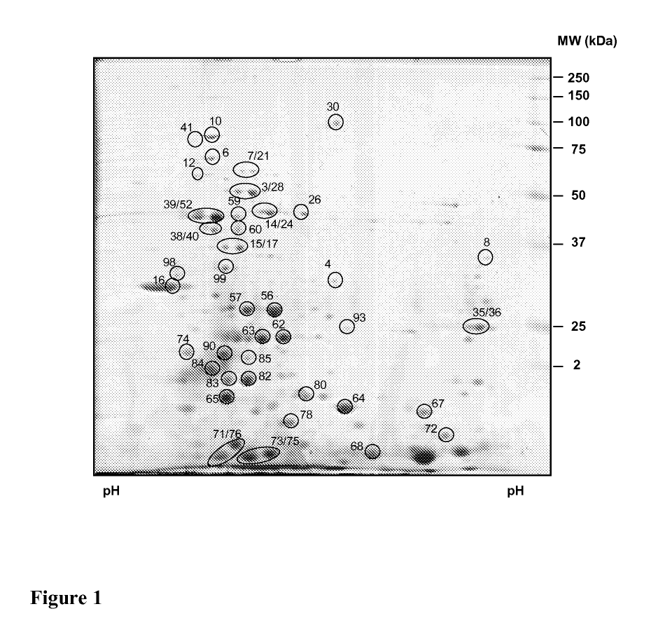

FIG. 1 shows a 2-dimensional, Coomassie blue stained SDS PAGE of a crude extract of Parachlamydia acanthamoebae. The extract was separated using a pH 3-11 NL IPG strip in the first dimension followed by a 12.5% SDS PAGE in the second dimension. Specific spots of antigenic peptides are encircled and numbered as described in the examples.

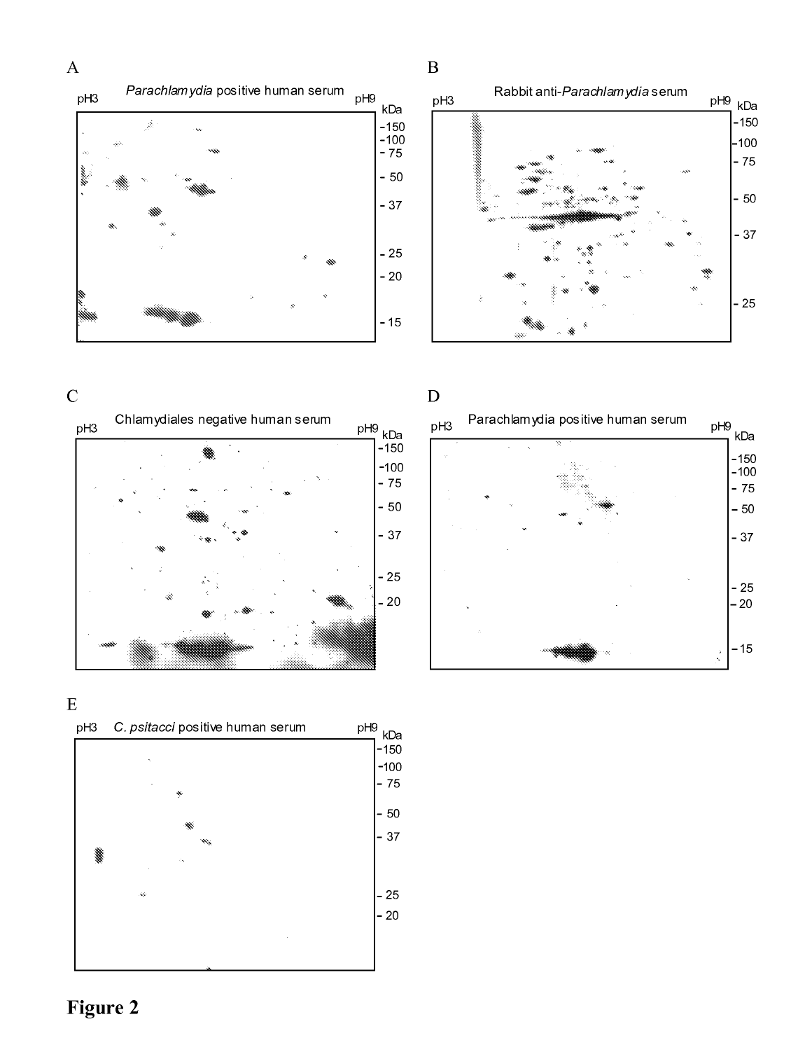

FIGS. 2A-E show western blots of parachlamydial proteins exposed to (A) and (D) sera of two human patients tested positive for Parachlamydia; (B) serum of a rabbit immunized with Parachlamydia; (C) serum of a human individual tested negative for Chlamydiales, and (E) serum of a human patient tested positive for infection by Chlamydia psittaci.

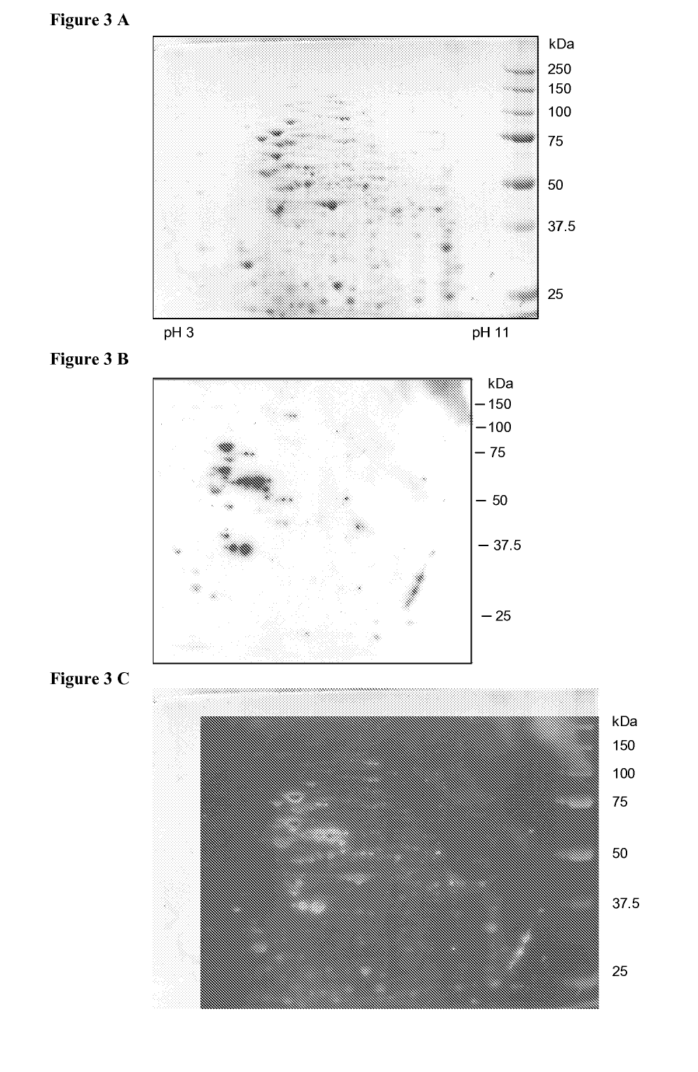

FIGS. 3A-C illustrate the method for detecting immunogenic proteins from a Chlamydia-like organism. FIG. 3A is a 2D Coomassie blue stained SDS PAGE of a Parachlamydia acanthamoebae proteome (similar to FIG. 1 but with spots not yet being numbered); FIG. 3B is a western blot obtained with human Parachlamydia positive serum; and FIG. 3C is the superposition of A and B, which allows to identify immunogenic proteins.

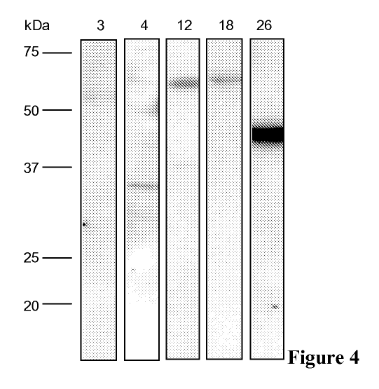

FIG. 4 shows western blots recognizing recombinant and purified proteins according to the present invention. The proteins correspond to spots 3, 4, 12, 18 and 26 in Table 1 and FIG. 1 (SEQ. ID. NO.:1, 2, 9, 16, and 17) and are blotted on nitrocellulose membranes probed with serum from an immunized rabbit.

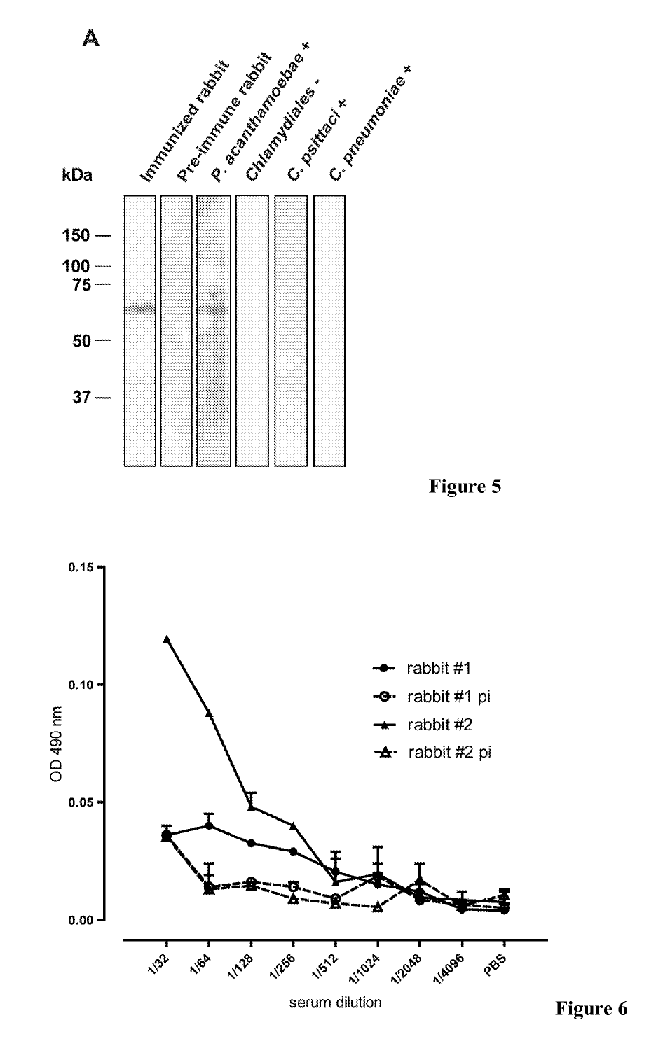

FIG. 5 shows western blots recognizing a recombinant and purified protein according to the present invention. The protein corresponds to spot Pac12 in Table 1 and FIG. 1 (SEQ. ID. NO.: 9) and is blotted on a nitrocellulose membrane and probed, in the first lane (from the left): with a serum from an immunized rabbit; in the second lane: with serum from the same rabbit before immunization; in the third lane: with a Parachlamydia positive serum (patient 3 in Table 1); in the fourth lane: with Chlamydiales negative serum (individual 12 (N-C9) in Table 1), in the fifth lane: C. psittaci positive serum (individual 18 in Table 1), in the lane on the far right: a C. pneumoniae positive serum (patient 15 in Table 1). Molecular mass standards (in kDa) are indicated on the left side. It can be seen that the protein is a good candidate for a serum-based diagnosis sensitive for Parachlamydia.

FIG. 6 shows the result of an ELISA conducted with serum at different dilutions of two rabbits immunized with the recombinantly produced and purified protein shown in FIG. 5 (SEQ ID NO: 9). Sera from 2 rabbits immunized with P. acanthamoebae and pre-immune (pi) sera were tested in duplicates.

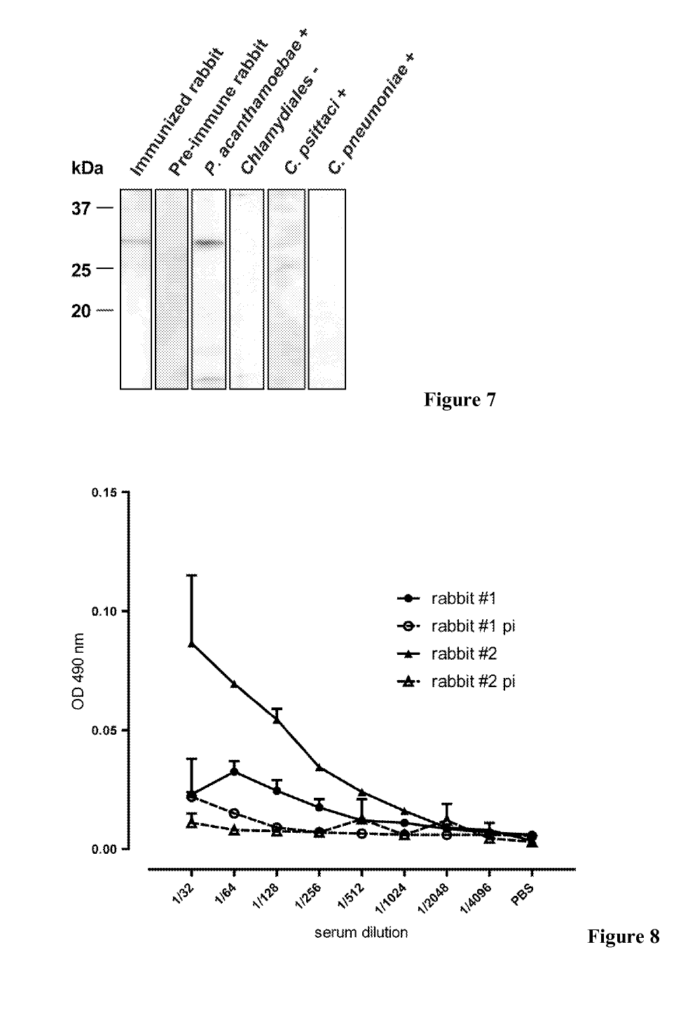

FIG. 7 corresponds to FIG. 5, but the protein corresponding to spot Pac4 (SEQ. ID. NO.: 2) in Table 1 and FIG. 1 was used.

FIG. 8 is the result of ELISA tests as described for FIG. 6, for which the protein corresponding to spot Pac4 (SEQ. ID. NO.: 2) in Table 1 and FIG. 1 was used.

### DETAILED DESCRIPTION OF THE PREFERRED EMBODIMENTS

The present invention relates to the at least one peptide for use in diagnosis. Preferably, diagnosis is the diagnosis for infection by a Chlamydia-like microorganism. As indicated above, the term “Chlamydia-like” or “Chlamydia-related” microorganism, for the purpose of the present specification, encompasses a large variety of strict intracellular bacteria belonging to the Chlamydiales order, but which exhibit enough biological differences with Chlamydiaceae to be assigned to other families.

According to a preferred embodiment, the Chlamydia-like microorganism belongs to the family of Prachlamydiaceae, more preferably to the genus of Parachlamydia, and most preferably it is Parachlamydia acanthamoebae.

The present invention relates to peptides, uses, tests, diagnosis and methods. These are all interrelated such that reference herein to a specific peptide, test or use, for example, automatically also refers also to the methods and diagnosis disclosed herein, and vice versa.

According to an embodiment, the diagnosis and or tests disclosed herein are specific. “Specificity”, for the purpose of the present specification, can be at the order level, at the family level, at the genus level and at the level of the species.

In one embodiment, the methods, tests and/or diagnosis as disclosed herein are specific at the level of the order, meaning that they allow diagnosis of infection by a given clade (i.e. any Chlamydiales), but does not yield a positive result when there is an infection, for example, of other Eubacteria. In other words, when the result of the diagnosis and/or the test is positive, it can be said that the positivity relates to an infection by a bacterial species of the order of the Chlamydiales, and not to an infection by a bacterium from another order. This does not exclude the possibility that an infection by such a bacterium of another order is also present, but it specifies that an infection by a bacterium that is part of this order is present.

According to an embodiment, the methods, tests and/or diagnosis disclosed herein are specific at the level of the family of the Parachlamydiaceae. In analogy of the before mentioned specificity with respect to the order, this means that the present invention allows the diagnosis of infection by any Parachlamydiaceae, but does not yield a positive result when there is an infection, for example, by a species belonging to the same order (Chalmydiales) but to another family than Parachlamydiaceae.

According to an embodiment, the methods, tests and/or diagnosis disclosed herein are specific at the level of the genus. In analogy to the paragraphs above, this means that the present invention allows the diagnosis of infection by any Parachlamydia, but does not yield a positive result when there is an infection, for example, of another genus of the family of the Parachlamydiaceae.

According to an embodiment, the methods, tests and/or diagnosis disclosed herein are specific at the level of the species any P. acanthamoebae. In analogy to the paragraphs above, this means that the present invention allows the diagnosis of infection by any strain belonging to the species of P. acanthamoebae, but does not yield a positive result when there is an infection, for example, of another species of the genus of Parachlamydia.

According to an embodiment, the diagnosis and/or the test disclosed herein are specific for an intracellular bacterial species.

Due to the possible role of Chlamydia-like microorganisms as causative agents of various conditions such as bronchitis and other mentioned further below, the present invention also relates to the diagnosis of these conditions and/or in the assessment of a risk and/or possibility of imminent or future contracting of one or more of these conditions.

A “peptide”, for the purpose of the present specification, refers to a genus of peptide or peptide fragments that encompass the amino acid sequences identified herein, as well as smaller fragments. A peptide is preferably defined in terms of its antigenic relatedness to any peptide disclosed herein. Thus, in one embodiment, a peptide within the scope of the invention is defined as an amino acid sequence comprising a linear or 3-dimensional epitope shared with any amino acid sequence disclosed herein.

According to a preferred embodiment, a peptide within the scope of the present invention is recognized by an antibody that specifically recognizes and/or binds to any peptide having an amino acid sequence as disclosed in the present specification. Antibodies are defined to be specifically binding if they bind peptides of the invention with Kₐ of greater than or equal to about 10⁷ M⁻¹, such as greater than or equal to 10⁸ M⁻¹.

The peptide may be a polypeptide (protein) or an oligopeptide, having from 3-10 amino acids. The size of the peptide is not crucial, as long as it has the minimum size for providing an antigenic entity and/or an epitope, which will be specifically targeted by the immune system of a human or animal subject. Preferably, a peptide comprises a continuous amino acid sequence of at least 10 amino acids, more preferably at least 15 and most preferably at least 20 amino acids.

The term “to comprise” or “comprising”, for the purpose of the present specification, is intended to mean “includes amongst other”. It is not intended to mean “consists only of”.

According to the invention, at least one peptide, as characterised by a specific amino acid sequence, is needed for the purpose of diagnosis, for example in the test of the present invention. Preferably, however, two or more different peptides are used, preferably three and most preferably four or more peptides. The use of a combination of a certain number of different, selected peptides reduces cross-reactivity and thereby enables to reduce the number of false positive and/or false negative diagnostic outcomes.

The peptide of the present invention is preferably a recombinant and/or an isolated peptide. A recombinant peptide can be produced by any suitable organism, for example a bacterium, an eucaryotic cell, a multicellular organism such as a transgenic plant or animal, for example. The recombinant peptide may be isolated or may not be. The recombinant peptide may, for example, be used in the form of an unpurified bacterial lysate, together with other cellular components.

The isolated peptide may be recombinant or may not be recombinant, for example isolated from the original organism. The peptide may be isolated according to any peptide and/or protein isolation and/or purification procedure.

The peptide is preferably selected from proteins with the amino acid sequences of SEQ. ID. NO.: 1-57. Amino acid sequences SEQ. ID. NO.:1-51 were selected on the basis of their antigenic properties. Epitopes present on these proteins are recognised by individuals tested positive for an infection by Parachlamydia. SEQ. ID. NO.: 52-57 are determined from DNA sequences selected from the genome of the P. acanthamoebae Hall coccus strain established for the purpose of the present invention (Example 5). The sequences SEQ. ID. NO.: 52-57 are thus selected by their sequence homology with sequences known to constitute immunogenic antigens in other organisms.

According to a preferred embodiment, the peptide comprises an amino acid sequence selected from: (a) an amino acid sequence selected from any one of SEQ. ID. NO.:1-57, preferably 1-51, (b) variant amino acid sequences having at least 50% of sequence identity with any one of the sequences of SEQ. ID. NO.: 1-57, preferably 1-51; and/or (c) fragments of the amino acid sequences of (a) and/or (b).

According to an embodiment, the peptide comprises (a) an amino acid sequence selected from any one of SEQ. ID. NO.: 1, 2, 6, 7, 9, 13, 14, 15, 17, 18, 19, 22, 23, 21, 35, 39, (b) variant sequences thereof, or (c) a fragment of (a) and/or (b).

According to preferred embodiment, the peptide comprises (a) an amino acid sequence selected from any one of SEQ. ID. NO.: 2, 6, 7, 9, 13, 14, 18, 23, 35, 39, (b) variant sequences thereof, or (c) a fragment of (a) and/or (b).

According to another preferred embodiment, the peptide comprises (a) an amino acid sequence selected from any one of SEQ. ID. NO.: SEQ ID NO: 2, 6, 9, 18, 39, (b) a variant thereof, or (c) a fragment of (a) and/or (b). Preferably, the peptide comprises an amino acid sequence selected from SEQ. ID. NO.: 2 and 9, variant sequences thereof, and/or a fragment of the sequence or of the variant sequence.

Table 1 below allows further selection of groups of preferred peptides for the purpose of the present invention. In particular, in Table 1, the following four categories of peptides can be distinguished, which represent advantageous properties for the purpose of the invention. Overlaps of peptides present in two, three or four of these categories result in more and most preferred

Category 1: Sequences of peptides recognized by at least one of the two immunized rabbits (a filled box in lane R1 and/or R2 of Table 1 below):

### SEQ. ID. NO.: 1-7, 9, 13-15, 17, 18, 19, 21-23, 25, 26, 28, 35, 37, 40, 43-47, 51.

Category 2: Sequences of peptides not recognized by serum of humans not tested positive (tested negative) for Chlamydiales in general (lack of a dark box in one of lanes 10-12 (B) in Table 1):

### SEQ. ID. NO.: 2, 3, 6-10, 14, 16, 17, 18, 24, 26, 28, 29, 33-37, 39, 40, 42-47, 51.

Category 3: Sequences of peptides not exhibiting any cross reaction with serum of any patient tested positive with any one of C. pneumoniae or C. psittaci (lack of a dark box in any one of lanes 13-17 (C-E)).

### SEQ. ID. NO.: 2, 3, 6, 8, 9, 10, 13, 16, 18, 24, 26, 28, 29, 32, 36, 39, 40, 42, 43, 45-51.

Category 4: Sequences of peptides recognized in at least one of the infected patients (presence of a dark box in at least one of lanes 1-9 (A)):

### SEQ. ID. NO.: 2-10, 13-19, 21-25, 35, 37, 40.

It is noted, with respect to different spots that turned out to represent the same protein and thus are present in several rows, with respect to categories 1 and 4, it is sufficient that one of the spots is recognized, and with respect to categories 2 and 3 (lack of box) that there need to be a lack in all spots (rows) referring to the same sequence.

Peptides falling in 1, 2, 3 or all 4 of these categories constitute preferred and most preferred groups of peptides, from which the one, two or more peptides may be selected for the purpose of the present invention.

### Overlaps:

Overlap of Cat. 1 and 2: SEQ. ID. NO.: 2, 3, 6, 7, 9, 14, 17, 18, 26, 28, 35, 37, 40, 43-47, 51.

Overlap of Cat. 1 and 3: SEQ. ID. NO.: 2, 3, 6, 9, 13, 18, 26, 28, 40, 43, 45-47, 51.

Overlap of Cat. 1 and 4: SEQ. ID. NO.: 2-7, 9, 13-15, 17-19, 21-23, 25, 35, 37, 40.

Overlap of Cat. 2 and 3: SEQ. ID. NO.: 2, 3, 6, 8-10, 16, 18, 24, 26, 28, 29, 36, 40, 42, 43, 45-47, 51

Overlap of cat. 2 and 4: SEQ. ID. NO.: 2, 3, 6-10, 14, 16, 17, 18, 24, 35, 37, 40.

Overlap of Cat. 3 and 4: SEQ. ID. NO.: 2, 3, 6, 8-10, 13, 16, 18, 19, 21, 24, 40.

Overlap of Cat. 1, 2 and 3: SEQ. ID. NO.: 2, 3, 6, 9, 18, 26, 28, 40, 43, 45-47, 51.

Overlap of Cat 1, 2 and 4: SEQ. ID. NO.: 2, 3, 6, 7, 9, 14, 17, 18, 35, 37, 40.

Most preferred group of peptides: Overlap of Cat. 1, 2, 3 and 4: SEQ. ID. NO.: 2, 3, 6, 9, 40.

Of course the peptide of the invention may comprise an amino acid sequence selected from any one of the groups and overlap groups of amino acid sequences defined above, variants thereof and fragments thereof.

The embodiments detailed above relate to preferred peptides as detailed in the examples, which are selected in order to make a diagnosis more specific with respect to the microorganism and/or more sensitive. They are also selected so as to avoid cross-reactivity.

The embodiments detailed above related to the preferred peptides relate to all peptides, compositions, uses, diagnosis, tests, assays and methods disclosed herein to which such peptides apply. For example, the preferred peptides are also preferred with respect to the method of preparing an antibody, as disclosed further below.

Instead of the peptides as defined by an amino acid sequences according to any one of SEQ. ID. NO.: 1-57, the peptide may comprise an amino acid sequence, which is a variant sequence of a sequence according to any one of SEQ. ID. NO.: 1-57 and/or of the preferred peptides as detailed above.

A peptide comprising a variant amino acid sequence, also referred to as variant peptide herein, means a peptide having to a large extent an identical amino acid sequence as the native and/or originally defined peptide, but which amino acid sequence is different from that disclosed herein because of one or more amino acids are removed, are substituted by one or more other amino acids and/or because further and/or other amino acids are added. Any combinations of substituted, deleted and added amino acids are possible for the purpose of defining a variant peptide.

Variants can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polyar residue for another, such as between Lys and Arg; Glu and Asp; or Gln and Asn. See Zubay, Biochemistry, Addison-Wesley Pub. Co., (1993). The effects of such substitutions can be calculated using substitution score matrices such as PAM-120, PAM-200, and PAM-250 as discussed in Altschul (J. Mol. Biol. 219:555-65, 1991). Other such conservative substitutions, for example, substitutions of entire regions having similar hydrophobicity characteristics, are also known. Naturally peptide variants are proteins that result from alternate mRNA splicing events or from proteolytic cleavage of the peptides described herein. Variants attribuatable to proeolysis include, for example, differences in the N- or C-termini upon expression in different types of host cells, due to proteolytic removal of one or more terminal amino acids from the polypeptides encoded by the sequences of the invention.

For the purpose of the present specification, “variant” peptides or sequences are defined by their sequence identity with respect to the original amino acid sequence. According to an embodiment, a variant peptide or amino acid sequence preferably is a at least 50%, 55%, 60%, 65%, 70%, 75%, 80%, 82%, 85%, 85%, 86%, 87%, 89%, 90%, 91%, 92%, 93%, 94%, 95%, 96%, 97%, 98% or 99% identical to any one of the amino acid sequences selected from any one of SEQ. ID. NO.: 1-57. The length of comparison sequences will generally be at least 10, 30, 30, 50, 100 or more amino acids. For example, the entire sequence can be compared.

In order to determine sequence identity with one of the reference sequences of the present invention, sequence identity comparison by blast using the basic protein blast on the internet (http://blast.ncbi.nlm.nih.gov) with preset standard parameters and database selections is used. This sequence comparison tools is based on algorithms detailed in the two following publications: Stephen F. Altschul, Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402. Stephen F. Altschul, John C. Wootton, E. Michael Gertz, Richa Agarwala, Aleksandr Morgulis, Alejandro A. Schäffer, and Yi-Kuo Yu (2005) “Protein database searches using compositionally adjusted substitution matrices”, FEBS J. 272:5101-5109.

Standard parameters include the selection of blastp (protein-protein BLAST, automatic adjustment of parameters to short input sequences; expect threshold 10, word size 3, use of the matrix BLOSUM62; Gap costs: existence: 11, extension 1; conditional compositional score matrix adjustment, no filters and no masking).

The peptides of the present invention also encompass fusion peptides and/or proteins. Fusions of additional peptide sequences at the amino and/or carboxyl terminal ends of the (poly)peptides of the invention may be used to enhance expression and/or extracellular secretion and/or may aid in the purification of the protein, for example. For example, peptides as defined herein further comprising a signal peptide and/or a his-tag or a different tag fulfilling any specific function are also encompassed by the present invention.

It is also possible to create a fusion protein containing a non-antigenic amino acid sequence combined with an amino acid sequence or fragment thereof as defined herein.

The peptide of the present invention may be a fragment of a polypeptide according to anyone of SEQ. ID. NO.: 1-57 disclosed herein, or of a variant peptide as defined above. A fragment peptide preferably has at least 10, 30, 40, 50, 60, 70, 80, 90, 100, 110, 120, 130, 140, 150 or more amino acids. In principle, any fragment of said sequences or variants is encompassed by the present invention. Since the peptide is used in diagnosis and preferably in an antibody-based, serological test, the fragment is preferably sufficiently large to be recognized by an antibody that specifically recognizes and/or binds to the fragment having an amino acid sequence as defined herein. The fragment of the invention may be used as such or as a stretch of amino acids in a larger polypeptide and/or amino acid sequence. For example, the antigenic fragment may be combined with, for example fused to, a non-antigenic amino acid sequence. According to an embodiment, the fragment has 30 or more, preferably 40 or more and most preferably 50 or more amino acids, wherein “or more” includes lengths of up to the entire protein.

It is important to note that certain stretches of the amino acid sequences disclosed herein (SEQ. ID. ID. NO.: 1-57) may exhibit similarity with stretches of sequences of proteins of other microorganisms of the order of the Chlamydiales. This presents a risk of cross-reactivity if only the respective stretch is considered. However, it is possible to identify stretches of amino acid sequences in such sequences that are not present in other Chlamydiales. Such stretches may thus be selected and isolated as a fragment by the skilled person and be used for a specific diagnosis or vaccination of/against a Chlamydia-like microorganism, such as a Parachlamydiaceae.

The present invention also relates to a test of diagnosis. The test is preferably a serological test, which means that a diagnosis can be made on the basis of a blood and/or serum sample of an individual. Preferably, a serum sample is used. Preferably, a sample comprises 5 ml or less of blood and/or serum of an individual, for example 0.5 ml or less. Preferably, the sample is diluted, the test being sufficiently sensitive to provide an accurate result with the diluted sample. For example, the dilution is at least ½, more preferably at least ¼, and most preferably at least ⅛. According to an embodiment, the serum is diluted to a dilution in the range of ⅛ to 1/256, more preferably 1/16 to 1/100. Within these ranges, the response is generally sufficiently strong to be distinguished from the serum of a non-infected human or animal individuals. Serum of specifically immunized animals may be further diluted, as can be seen from the examples below.

The test is preferably an antibody-based test, which means that the presence of antibodies specifically binding to a peptide as defined herein, said antibody being contained in the blood and/or serum sample of an individual, is detected, and from the presence of a sufficient amount of such antibody. The test may also contain one or more antibodies that specifically binds to a peptide as defined herein.

According to an embodiment, the test of the invention comprises a support, such as a microtiter plate, a glass strip or another carrier material, on which at least one peptides as defined herein is provided. The support may be then exposed to diluted serum, for example.

An example for a diagnostic test of the present invention is an Enzyme-Liked Immunosorbent Assay (ELISA). Another example are immunofluorescent strips as marketed by the company inoDiag in 83 870 Signes, France, under the trademark InoMu.S.T.®. These glass strips contain a spot, which is incubated with a diluted blood sample and with further reactants generally comprising an antibody selective for the constant domains of a specific human antibody type. Subsequently, the strip is fluorescence analysis in an automated process and the diagnosis is accomplished. The incubation with serum as well as the reading of the fluorescent signal and its interpretation is generally done in an automated process. The present invention thus encompasses this type of serological test, in which antigenic peptides of a Chlamydia-like microorganism, in particular a microorganism belonging to the Parachlamydiaceae, in particular the peptides of the invention, are used. These types of serological tests are disclosed, for example, in Gouriet et al., “Comparison of the new InoDiag automated fluorescence multiplexed antigen microarray to the reference technique in the serodiagnosis of atypical bacterial pneumonia”, CMI, 2008, 14, 1119-1127, and Gouriet et al., 2008, CMI, 14, 1112-1118.

According to another embodiment, the test of the invention is based on the detection of an antigen.

For example, the test of the present invention may be a urine-based test. Such a test comprises, for example, an antibody that binds to antigens possibly present in the urine. The kit may contain a second antibody recognizing the first antibody and containing a reporting mechanism or system that permits the detection of a binding between the first antibody and the antigen, exploiting the same general principle as the ELISA test above, with the difference that the presence of an antigen instead of an antibody is detected. Variations of this pattern are possible. For example, a first antibody could be provided on a column, binding Parachlamydiaceae antigens, if present, in a urine sample. Recognition of the presence of the bound antigen can be made as is conventional, for example by adding a second antibody that is also specific to the antigen, but preferably to a different epitope, which second antibody again comprises a conveniently detectable reporting system.

Such a kit thus preferably comprises at least one antibody of the present invention, specific for the recombinant and/or isolated peptides of the invention. Such a kit preferably comprises a second antibody, which specifically binds to the first antibody or to the same antigen as the first antibody, but preferably to a different epitope.

The use, test and the method of the present invention preferably comprises one or more isolated and/or recombinant immunogenic peptides of an intracellular bacterial strain of the genus Parachlamydium, preferably of the species P. acanthamoebae, and most preferably of the Hall P. acanthamoebae coccus strain.

The present invention also relates to a method of producing an antibody, the method comprising the steps of exposing a mammal to a peptide as disclosed herein, and, following exposure, harvesting polyclonal antibodies from the serum of said mammal. The peptide may be administered to the mammal as an isolated peptide, as a recombinant peptide, as an unpurified bacterial extract containing the peptide, for example, an unpurified lysate and/or bacterial extract of recombinant bacteria expressing said peptide. Preferably, one, two, three, four, five or more recombinant peptides are used for immunisation of the mammal. Preferably, the peptides or any composition comprising the peptides are administered by injection, for example subcutaneous injection. The peptide may be administered in the form of a composition, for example an aqueous solution comprising the peptide or a mixture of different peptides as defined and disclosed herein.

The present invention also provides a monoclonal antibody, wherein said antibody specifically binds to a peptide as defined herein, and also to a hybridoma producing the antibody. Methods for producing a hybridoma from myeloma cells and cells isolated from immunized individuals, such humans or animals, in particular mammals, in particular mice, rabbits, goats, for example, are known. With respect to the present invention, a mammal is immunized with an isolated and/or recombinant peptide as defined herein, or composition of two or more different peptides as defined herein. Specifically binding, for the purpose of the present invention, may be determined by immunofluorescence, western blotting, ELISA and/or immunohistochemistry, while using the corresponding antigen.

According to an embodiment, the antibody of the invention preferably specifically binds to one of the peptides as disclosed and defined in the present specification. Preferably, the antibody does not bind to peptides or proteins of microorganisms of Chlamydiales that are other than Parachlamydiaceae. In particular, the antibody does not bind to one or more proteins expressed by C. pneumonia, C. trachomatis and C. psittaci.

The polyclonal and/or monoclonal antibodies referred to herein are preferably isolated antibodies.

The antibodies of the present invention may be used in immunohistochemical methods, assays and/or tests for detecting a Chlamydia-like microorganism in tissue, in particular tissue samples. These methods, assays and/or tests may comprise primary and secondary antibodies, wherein the primary antibody is the antibody of the present invention, and a secondary antibody is provided that specifically binds to said primary antibody and the presence of which secondary antibody can be visualized, for example by linking an enzyme to said secondary antibody and/or a fluorescent dye, as is conventional in immunohistochemistry.

The tissues and/or tissue samples mentioned herein are preferably prepared and/or treated so as to disrupt cell membranes, of the tissue and thereby expose antigens possibly present in the tissue. The tissues may also be treated so as to unmask the antigens in order to allow for better antibody-antigen recognition.

The present invention also relates to vaccines, methods of vaccination and pharmaceutical compositions. For example, the pharmaceutical composition may be a composition for the prophylaxis of a disease or infection, in particular of an infection by a Chlamydia-like microorganism such as a Parachlamydiaceae. A vaccine composition is an example of a pharmaceutical composition.

The products, compositions, test kits for diagnosis, vaccines, etc. disclosed herein preferably comprise at least two different peptides as disclosed and/or defined herein. The use of several peptides may, in the case of a vaccine, increase the occurrence of a protective immune reaction. In the case of diagnosis, the use of two or more different peptides may increase reliability, in particular the avoidance of false negative outcomes. According to an embodiment at least two, preferably at least three, more preferably at least four and most preferably at least five different immunogenic peptides as disclosed and/or defined herein are used.

Parachlamydiaceae, such as P. acanthamoebae are possibly involved and/or causative agents for various conditions, such as lower and/or upper respiratory tract infections, for example infections of the nasal concha, bronchitis, bronchiolitis and/or pneumonia, conjunctivitis, uveitis, infections of the urogential tract, kidney infection, pericarditis, infertility, they may cause abortion and pre term labour in animals and miscarriage and pre term labour in humans, amongst other conditions.

The antigenic peptides of the invention may be used in the diagnosis of the above conditions, to assess the risk and/or possibility of imminent or future contracting of one or more of these conditions and, in particular, to find if a Chlamydia-like organism is at the origin of the condition. Furthermore, the vaccines and/or methods of vaccination of the present invention may be used prevent and/or treat the above-mentioned conditions.

The above mentioned conditions may appear in humans or animals. According to an embodiment, the vaccine is destined to humans. According to another embodiment, the vaccine of the invention may be used in particular to treat one or more of these conditions in animals, for example domestic animals, in particular livestock. For example, the vaccine is used in the prevention and/or treatment of one or more of said conditions in ruminants, such as cattle, sheeps, goats, and the like.

The invention is now illustrated by way of the examples below, which are not intended do limit the scope of the present invention.

### EXAMPLES

### Patients

In the publication of G. Greub, I. Boyadjiev, B. La Scola, D. Raoult and C. Martin, “Serological Hint Suggesting That Parachlamydiaceae Are Agents of Pneumonia in Polytraumatized Intensive Care Patients”, Ann. N.Y. Acad. Sci. 990: 330-319 (2003) sera taken of intensive-care patients and from healthy blood donors were tested for reactivity against Parachlamydia by immunofluorescence. On pages 312-313 the determination of infection by detection of specific antibodies against Parachlamydia is described in detail under “Serology”. In five patients, infection by Parachlamydia was found. Sera of these five patients, and of two additional patients, in which anti-Parachlamydia antibodies could be identified in the same manner, were used in the examples below. In addition, sera were also taken from women with miscarriage and at term uneventful pregnancy (Baud et al. Emerg. Infect. Dis. 2007, 13 (8), 1239-1243). Using a similar approach than that used in the above mentioned publication by Greub et al., infection by Parachlamydia acanthamoebae was documented in 7 patients. No cross-reactivity was found in sera taken from both work mentioned above.

### Example 1

### Cultivation and Purification of Parachlamydia acanthamoebae

A procedure for purifying P. acanthamoebae is described by G. Greub, J.-L. Mege and D. Raoult “Parachlamydia acanthamoebae Enters and Multiplies within Human Macrophages and Induces Their Apoptosis”, Infection and Immunity, October 2003, p. 5979-5985, more particularly on page 5979 under the title “Materials and methods” the paragraph “P. acanthamoebae culture and purification”. This procedure was used with the exception that instead of A. polyphaga, the A. castelanii strain ATCC 30010 was used as a host. Furthermore, there was no tittering, lysis test and freezing conducted. Instead, the large lower band of Parachlamydia resuspended twice in PBS was subjected to 2D gel electrophoresis as reported below.

### Example 2

### Crude Extract Sample Preparation and 2-D Gel Electrophoresis

Purified bacteria (mostly elementary bodies, EB) were washed twice in 10 mM Tris, 5 mM MgAc, pH 8.0 and then lysed by 5 cycles of short-pulse sonication in lysis buffer (30 mM Tris, 7M urea, 2M Thiourea, 4% CHAPS, pH 8.5). Proteins were recovered by centrifugation at 8′0000 rpm and quantified using a Bradford assay (Quick Start™ Bradford Protein Assay, Bio-Rad laboratories, Hercules, USA). Routinely about 4 mg of total parachlamydial proteins were obtained from 60 T75 flasks of amoebal co-culture. Aliquots of 1.2 mg were stored at minus 80° C. for subsequent electrophoretic analysis.

Two dimensional gel electrophoresis was performed as described by Centeno et al (Centeno et al., Cell Death and Differentiation, 2007, 14, p. 240-253) using approximately 150 μg (mini gels) or 600 μg (midi-gels) of total EB proteins for each electrophoretic run. Proteins were visualized by Coomassie Blue staining or transferred to nitrocellulose. FIG. 1 shows the 2D gel obtained.

### Example 3

### Immunoblot Analysis

Nitrocellulose membranes were blocked by 2 hours incubation with 5% non-fat dry-milk in Tris-buffered saline with 0.05% Tween 20 (TBS), washed 3 times with TBS, 0.5% milk and incubated overnight at 4° C. with sera diluted in TBS, 0.5% milk. Membranes were probed either with human sera (see “Patients” above (dilution 1/64) or with sera (dilution 1/25) of rabbits immunized 4 times with purified and heat-inactivated bacteria (Eurogentec standard protocol). After 3 subsequent washes with TBS, 0.5% milk, the membranes were probed with horseradish peroxidase-conjugated goat anti-human IgG (Chemicon, Temecula, Calif., 1:5000), or anti-rabbit IgG (Cell Signaling, Allschwill, Switzerland, 1:1000). Membranes were then washed 3 more times with TBS and immunoreactive spots were detected with a chemiluminescence-based kit (LiteAblot™, Euroclone SpA, Pero, Italy).

FIG. 2 shows the result of immunoblot analysis obtained with sera as indicated. Serum of a patient negative by IF for all Chlamydiae tested: This blot shows cross-reactions with similar proteins in other organisms (par ex: ribosomal protein L7/L12, DnaK, HSP 60).

### Example 4

### Selection of Immunogenic Proteins (Antigens)

The Coomassie Blue stained gel and the immunoblots obtained after incubation with P. acanthamoebae positive sera or rabbit anti-P. acanthamoebae sera were overlapped using the Adobe Photoshop program to select spots corresponding to immunoreactive proteins.

This process is illustrated in FIGS. 3A-C, with FIG. 3A being the 2D PAGE with Parachlamydia Comassie blue stained proteins and FIG. 3B is a western blotting obtained with human Parachlamydia positive serum. FIG. 3C is the overlap of FIGS. 3A and B, wherein, in the overlap, the spots obtained with human serum appear in blue.

The overlap procedure allows to differentiate sports of interest of the Parachlamidia proteome from non-antigenic spots. The spots of interest (antigen spots) were marked and numerated, as illustrated in FIG. 1.

Table 1, annexed further below, contains the result of the overlap analysis of immunoblotting obtained with serum of different patients and immunized rabbits exposed to the western blot of the P. acanthamoebae proteome. The black filled cells in Table 1 indicate that the respective patient and/or rabbit produced an antibody against the respective protein, referred to by its spot number. Table 1 also lists results obtained with serum of patients that were not tested positive for P. acanthamoebae, but which were positive for other Chlamydiales, in particular Chlamydia psittaci and/or Chlamydia pneumoniae. Furthermore, also the serum of individuals being negative for any Chlamydia-like organism was tested. The results of Table 1 thus allow identifying antigenic proteins of P. acanthamoebae that avoid cross-reactivity when exposed to serum of an individual infected with C. psittaci and C. pneumoniae.

Table 2 lists a selection of spots and the corresponding proteins as identified according to the procedure below. The listed antigenic proteins were recognized by serum of a rabbit immunized with P. acanthamoebae. For a test specific to P. acanthamoebae, spots are considered as “best/good candidates” when they were not recognized by serum of patients infected with C. pneumoniae or C. psittacci and are also not recognized by two control sera of patients negative for all Chlamydiales tested. These spots are listed on top in Table 2. These proteins are thus particularly useful in a serological test for infection by Parachlamydia.

TABLE 2
Evaluation of some identified P. acanthamoebae immunogenic
proteins for use in a serological diagnostic test.

| | | SEQ | |
|---|---|---|---|
| | | ID | |
| Comments | Spot | NO: | Protein description |
| Best candidates | 12 | 9 | Protein of unknown function |
| | 4 | 2 | Protein of unknown function |
| Other good candidates | 30 | 18 | Putative manganese and iron |
| | | | superoxide dismutase |
| | 8 | 6 | Putative NAD(P)H-dependent |
| | | | glycerol-3-phosphate |
| | | | dehydrogenase |
| | 74 | 39 | Putative yciF protein |
| Similarity with proteins | 10 | 7 | Molecular chaperone DnaK |
| of other species | 41 | 23 | 30S ribosomal protein S1 |
| | 6 | 4 | Chaperonin GroEL |
| | 73 | 37 | Probable 50S ribosomal protein |
| | | | L7/L12 |
| | 14 | 13 | Elongation factor Tu |
| | 15 | 14 | Elongation factor Ts |
| | 71 | 35 | Co-chaperonin GroEs |
| Cross reaction with | 10 | 7 | Molecular chaperone DnaK |
| C. pneumoniae, | 41 | 23 | 30S ribosomal protein S1 |
| C. psitacci or | 6 | 4 | Chaperonin GroEL |
| negative controls | 73 | 37 | Putative 50S ribosomal protein |
| | | | L7/L12 |
| | 3 | 1 | Putative serine proteinase |
| | 14 | 13 | Elongation factor Tu |
| | 26 | 17 | Protein of unknown function |
| | 52 | 21 | Protein of unknown function |
| | 40 | 22 | Protein of unknown function |
| | 15 | 14 | Elongation factor Ts |
| | 16 | 15 | Putative mip |
| | 36 | 19 | Protein of unknown function |

### Example 5

### Raw Genome Sequences Data and ORFing

Recent advances in DNA sequencing such as 454 pyrosequencing and Solexa technologies provide much faster, and much better sequencing strategies. We thus use the GS20 sequencer (454 Life Sciences, Brandford, USA) to sequence the genome of Parachlamydia acanthamoebae strain Hall coccus. Two independent runs were performed to increase the coverage. In order to correct possible frame shifts due to homopolymers, we also sequenced the Parachlamydia genomic DNA using the Solexa technology in Illumina Genome analyzer (Illumina Inc, San Diego, USA). The assembly of all GS20 sequences was done using the Newbler software (Roche, Basel) with default parameters except for overlap size (45 nucleotides) and identity score (95%). Solexa sequences were assembled using the Edena software (Hernandez et al. Genome research 2008). Both assemblies were then merged by reassembling the GS20 sequences with contigs of Solexa' assembly with Newbler. Remaining differences were manually inspected and corrected when necessary.

Then, ORFing was performed using Glimmer v3.02 trained on published sequences of all published genomes of Chlamydiales. These ORFs were then used as input in the Mascot program to look for mass spectrometry hits (see below).

### Example 6

### Protein Identification by MALDI-MS/MS

The antigen spots were excised from the 2-D gel and subjected to mass spectrometry (MS) to definitively identify the proteins.

Spots excised from the 2-D gel were transferred to special 96-well plates (Perkin Elmer Life Sciences). In-gel proteolytic cleavage with sequencing-grade trypsin (Promega, Madison, Wis., USA) was performed automatically in the robotic workstation Investigator ProGest (Perkin Elmer Life Sciences) according to the protocol of Shevchenko et al. (Shevchenko A, Wilm M, Vorm O, Mann M. Mass spectrometric sequencing of proteins silver-stained polyacrylamide gels. Anal Chem. 1996 Mar. 1; 68(5):850-8.). Digests were evaporated to dryness and resuspended in 3 ul alpha-cyano-hydroxycinnamic acid matrix (5 mg/ml in 60% (v:v) acetonitrile:water), of which 0.7 μl were deposed in duplicate on a target plate.

MALDI-MS-MS analysis was performed on a 4700 Proteomics Analyser (Applied Biosystems, Framingham, Mass., USA). After MALDI-TOF MS analysis, internal calibration on trypsin autolysis peaks and subtraction of matrix peaks, the 10 most intense ion signals were selected for MS/MS analysis. Non-interpreted peptide tandem mass spectra were used for direct interrogation of P. acanthamoebae contig database (3533 ORFs) using Mascot 2.0 (http://www.matrixscience.com). The mass tolerance for database searches was 50 ppm. MASCOT was set up to only report peptide matches with a score above 14. With the parameters used, the threshold for statistical significance (p<0.05) corresponded to a total (protein) MASCOT score of 17, but we considered only scores greater than 30. Proteins scoring above 80 were considered automatically as valid, while all protein identifications with a total MASCOT score between 30 and 80 were manually validated. Validation included examination of the peptide rms mass error of individual peptide matches. MS/MS Peptide matches were validated only if at least an ion series of 4 consecutive y ions were matched, in addition to ions belonging to other series.

### Example 7

### Cloning of Selected Immunogenic Proteins

The ORFs corresponding to the immunogenic proteins Pac1, Pac4, Pac12, Pac18, Pac26, Pac35 and Pac123 (SEQ. ID. NO.: 1, 2, 9, 16, 17, 19, and 55, respectively) were amplified by PCR using P. acanthamoebae DNA as template. The following primers were used:

Pac1 forward:
(SEQ ID NO: 58)
5′cagcatatgaattttaaatcttcctttaca3′
Pac1 reverse:
(SEQ ID NO: 59)
5′gatgagctcttaatcgactttaatagaaacgaag3′
Pac4 forward:
(SEQ ID NO: 60)
5′gatcatatggctataagtttatattcaaatcaa3′
Pac4 reverse:
(SEQ ID NO: 61)
5′gatggatccttaggtattagacgttgcaggttt3′
Pac12 forward:
(SEQ ID NO: 62)
5′gatcatatgacttacaataataatatcaatgtat3′
Pac12 reverse:
(SEQ ID NO: 63)
5′gatggatccctacggaatttgatcggaacgtt3′
Pac18 forward:
(SEQ ID NO: 64)
5′gattcatgagtccagatccaatcaaaggtttcg3′
Pac18 reverse:
(SEQ ID NO: 65)
5′gatctcgagctgtgtaagtccttggaacatct3′
Pac26 forward:
(SEQ ID NO: 66)
5′gaacatatgaatattaatagtactccacctg3′
Pac26 reverse:
(SEQ ID NO: 67)
5′gtcggatccttagtgaatacgataattaaatgcgc3′
Pac35 forward:
(SEQ ID NO: 68)
5′gatcatatgcaacgatcccttgggggt3′
Pac35 reverse:
(SEQ ID NO: 69)
5′gatggatccttagtattggctaccgcagcaa3′
Pac123 forward:
(SEQ ID NO: 70)
5′gactcatgagtaaaaaatggcatgtgattttatacg3′
Pac123 reverse:
(SEQ ID NO: 71)
5′gacctcgagaatgcgtttaagatgctgttgca3′

PCR products were cloned into the pET28 vector (Novagen EMD Chemicals Inc, San Diego, USA). This vector system allows the protein of interest to be expressed as a fusion protein with a 6 His tail fused to its N-terminus (Pac1, Pac4, Pac12, Pac18, Pac26, Pac35) or C-terminus (Pac123).

### Example 8

### Expression and Purification of Selected Proteins

Protein expression in E. coli DE-3 is induced with isopropyl-β-D-thiogalactopyranoside (IPTG, Qbiogen, Basel, Switzerland), in most of the cases with 1 mM IPTG during 2.5 hours at 37° C., but induction conditions must be adapted for each individual protein. The P. acanthamoebae proteins can be used as antigens in detection or diagnostic assays either as unpurified E. coli lysate or, for increased sensitivity and reduced background, as a purified product.

For purification, the bacterial pellet is resuspended in lysis buffer (100 mM NaH₂PO4 H₂O, 10 mM Tris, 8M urea, pH 8.0 (denaturing conditions) or 50 mM NaH₂PO4 H₂O, 300 mM NaCl and 10 mM imidazole, pH 8.0 (non denaturing conditions)) containing 1 mg/ml lysozyme, 1× Halt Protease Inhibitor (Pierce Biotechnology Inc, Rockford, USA), 5 μg/ml DNAseI and 8 μg/ml RNAseA and lysed by short pulses of sonication on ice. Protein samples are applied on a Ni-NTA column (HIS-Select™ Spin Columns, Sigma-Aldrich, Missouri, USA). The column is washed 2 times with washing buffer (100 mM NaH₂PO4 H₂O, 10 mM Tris, 8M urea, pH 6.3 (denaturing conditions) or 50 mM NaH₂PO4 H₂O, 300 mM NaCl and 20 mM imidazole, pH 8.0 (non-denaturing conditions)). And finally, the protein of interest is eluted from the column either by decreasing the pH of the solution to pH 5.9 and then to pH 4.5 (denaturing conditions) or by adding 250 mM imidazole to the last solution (non denaturing conditions). The most pure protein fractions are pooled and if necessary (denaturing conditions) dialysed to remove urea against 1×PBS containing 4M, 2M, and no urea at 4° C. Some recombinant proteins precipitate when urea concentration decreases. In these cases, urea concentration was kept high enough to ensure solubility of the protein (usually 2M).

The purified recombinant proteins are submitted to SDS PAGE and transferred to nitrocellulose membrane before probing with relevant mouse, rabbit or human sera or are used directly as antigens in serological tests. In FIG. 4, western blots of the recombinant and purified proteins of spots 3, 4, 12, 18 and 26 in Table 1 and FIG. 1 (SEQ. ID. NO.: 1, 2, 9, 16, and 17) probed with serum from an immunized rabbit are shown. FIGS. 5 and 7 show the recombinant protein Pac12 (SEQ. ID. NO.: 9) and Pac4 (SEQ. ID. NO.: 2) probed with various sera as indicated. Lack of cross-reactivity with serum of patients as indicated is demonstrated.

### Example 9

### Enzyme-Linked Immunsorbent Assay (ELISA)

Two of the 6His tail-purified proteins (Pac4 and 12, SEQ. ID. NO.: 2 and 9, respectively) were used in an ELISA. 96-well ELISA microplates were coated with 100 ng of purified proteins (Pac4, Pac12) in carbonate buffer pH 9.6 and incubated overnight at 4° C. After blocking with 3% non-fat dry-milk in PBST (PBS+0.1% Tween 20) during 1 hour at 37° C., plates were washed with PBST and incubated 2 hours at 37° C. with serial two-fold dilutions, in PBST+1% non-fat dry-milk, of sera from 2 rabbits immunized with P. acanthamoebae and of corresponding pre-immune sera. After 3 subsequent washes with PBST, plates were incubated 1 hour at 37° C. with horseradish peroxidase-conjugated anti-rabbit IgG (Cell Signaling, Allschwil, Switzerland) diluted 1:1000 in PBS+1% non-fat dry-milk. Plates were washed 5 more times with PBST. O-phenylenediamine dihydrochloride (OPD) in citrate buffer was used as substrate for the peroxydase. After 15 minutes incubation, the optical density was read at 492 nm using an ELISA reader (Multiskan Ascent, Thermo Scientific, Waltham, USA).

The result is seen in FIGS. 6 and 8 for proteins Pac12 and 4, SEQ. ID. NO.: 9 and 2, respectively.

TABLE 1
Reactivity of serum of individuals and rabbits towards Parachlamydia proteins

| | A | | | | | | | | | B | | | C | | | D | E | F | | | | | | |
|---|---|---|---|---|---|---|---|---|---|---|---|---|---|---|---|---|---|---|---|---|---|---|---|---|
| spot | 1 | 2 | 3 | 4 | 5 | 6 | 7 | 8 | 9 | 10 | 11 | 12 | 13 | 14 | 15 | 16 | 17 | R1 | R2 | R3 | R4 | R5 | R6 | SQ |
| Pac1 | | | | | | | | | | | | | | | 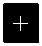 | | | 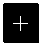 | | 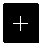 | | | +? | 1 |
| Pac2 | | | | | | | | | | | | | | | | | | | | | | | | 1 |
| Pac3 | | | | | | | | | | 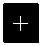 | | 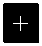 | | | | | | 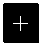 | | | | | | 1 |
| Pac4 | | 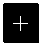 | | | | | | | | | | | | | | | | 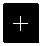 | | | | | | 2 |
| Pac5 | 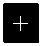 | | | 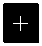 | | | 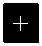 | 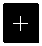 | | | | | | | | | | 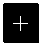 | | | | | | 3 |
| Pac6 | 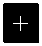 | 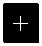 | 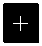 | 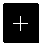 | 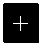 | 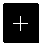 | | 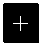 | | | 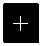 | | | 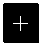 | | | | 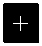 | 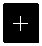 | 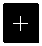 | | | +? | 4 |
| Pac7 | 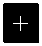 | 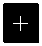 | | 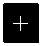 | 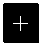 | 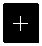 | | 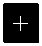 | | | | 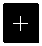 | | 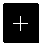 | | | | 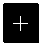 | | | | 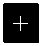 | 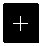 | 5 |
| Pac8 | 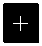 | | | 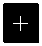 | | | | | | | | | | | | | | 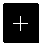 | | | | | | 6 |
| Pac9 | 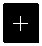 | | | 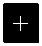 | | 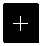 | | | | | 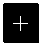 | | | | | | | 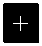 | | | | | | |
| Pac10 | | 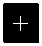 | | | 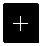 | 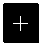 | 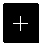 | 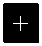 | 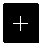 | | | | | 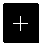 | | 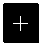 | | 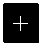 | 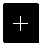 | | 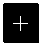 | | +? | 7 |
| Pac11 | | 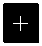 | | | | | | | | | | | | | | | | | | | | | | 8 |
| Pac12 | | 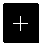 | | | | | 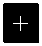 | | | | | | | | | | | 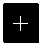 | | | | | 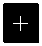 | 9 |
| Pac13 | | 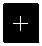 | | 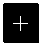 | | | | | | | | | | | | | | | | | | | | 10 |
| Pac14 | | 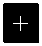 | 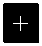 | 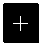 | 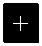 | 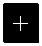 | 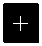 | 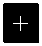 | 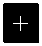 | | | 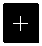 | | | | | | 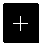 | 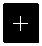 | | | 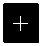 | | 13 |
| Pac15 | | | 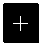 | | 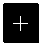 | 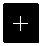 | 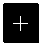 | | 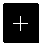 | | | | | 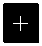 | 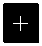 | 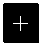 | 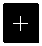 | 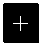 | | | | | 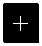 | 14 |
| Pac16 | | 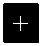 | | | | | | | | 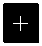 | | 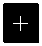 | | | | 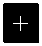 | | 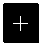 | | | | | 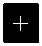 | 15 |
| Pac17 | | 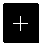 | | | 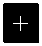 | 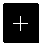 | | | 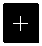 | | | | | 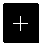 | | | | | 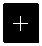 | | | | 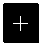 | 14 |
| Pac18 | 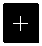 | | | | 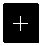 | | | 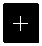 | | | | | | | | | | | | | | | | 16 |
| Pac19 | 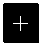 | | | | 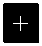 | | | 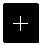 | | | | | | | | | | | | | | | | 16 |
| Pac20 | | 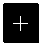 | | 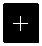 | | | | 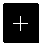 | | | 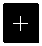 | | | | | | | | | | | 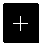 | 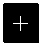 | |
| Pac21 | | 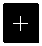 | | 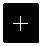 | 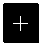 | 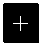 | | 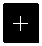 | | | | | | 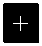 | | | | 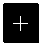 | 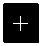 | | | 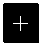 | 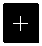 | 5 |
| Pac22 | 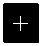 | | | | | | | | | | | | | | | | | | | | | | | |
| Pac23 | 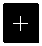 | | | | | | | | | | | | | | | | | | | | | | | |
| Pac24 | | | 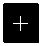 | 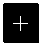 | | 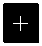 | 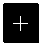 | 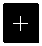 | 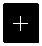 | | | | | | | | | | | | | 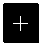 | | 13 |
| Pac25 | 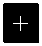 | | | | | 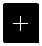 | | | | | 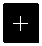 | | | | | | | 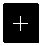 | | | | | | |
| Pac26 | | | | 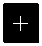 | | 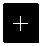 | | 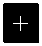 | 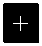 | | | | | 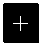 | 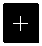 | 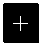 | | 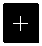 | 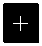 | | | | | 17 |
| Pac27 | | | | 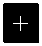 | | | | | | | | | | | | | | | | | | | | |
| Pac28 | | | | | | | 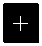 | | | | | | | | 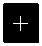 | | | | | 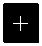 | | | +? | 1 |
| Pac29 | | | | | 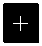 | | | | | | | | | | | | | | | | | | | |
| Pac30 | | | | | 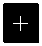 | 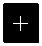 | | 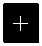 | | | | | | | | | | 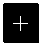 | | | | | | 18 |
| Pac31 | | | | | 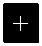 | 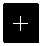 | | 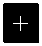 | | | | | | | | | | | | | | | | |
| Pac32 | | | | | 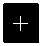 | | | | | | | | | | | | | | | | | | | |
| Pac33 | | | | | | 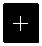 | | | | | | | | | | | | | | | | | | |
| Pac34 | | | | | | 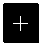 | | | | | | | | | | | | 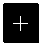 | | | | | | |
| Pac35 | | 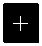 | | | 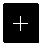 | 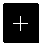 | | | | | | | | | | | | 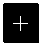 | | | | | | 19 |
| Pac36 | | | | | | | | | 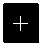 | | | 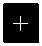 | | 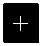 | | | | | | | | | | 19 |
| Pac37 | | | | | 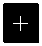 | | | | 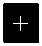 | 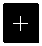 | | | | | 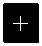 | | | | 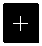 | | | | | 21 |
| Pac38 | | | | | 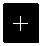 | | | | 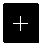 | | | 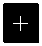 | | 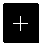 | 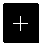 | | | | | | | | | 22 |
| Pac39 | | | | | | | | | 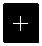 | | | 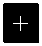 | | | | | | | 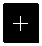 | | | 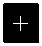 | +? | 21 |
| Pac40 | | | | | 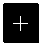 | | | | 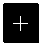 | | 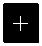 | 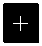 | | 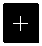 | | | 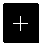 | 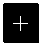 | 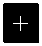 | | | 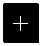 | | 22 |
| Pac41 | | | | | | 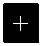 | | 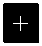 | | | | | | 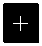 | 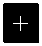 | 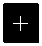 | | 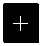 | 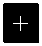 | | | | | 23 |
| Pac42 | | | | | | | | 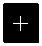 | 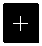 | | | | | | | | | | | | | | +? | |
| Pac43 | | | | | | 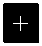 | | | 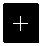 | | | | | | | | | | | | | | | |
| Pac44 | | | | | | 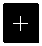 | 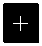 | | | | | | | | | | | | | | | | +? | 24 |
| Pac45 | | | | | | | 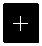 | 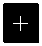 | | | | 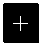 | | 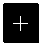 | | | | 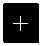 | | | | | +? | 25 |
| Pac46 | | | | | | | | 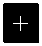 | | | | | | | | | | | | | | | | |
| Pac47 | | | | | | | | 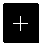 | | | | | | | | | | | | | | | | |
| Pac48 | | | | | | | | 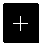 | | | | | | | | | | 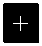 | | | | | | |
| Pac49 | | | | | | | | 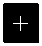 | | | | | | | | | | 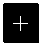 | | | | | | |
| Pac50 | | | | | | | | 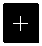 | | | 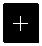 | | | | | | | 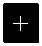 | | | | | | |
| Pac52 | 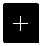 | 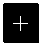 | 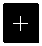 | | | | | | 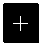 | | 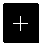 | 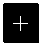 | | 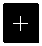 | 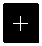 | | 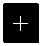 | | 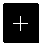 | | | | +? | 21 |
| Pac53 | | | | | | | | | | | | | | | | | | 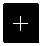 | | | | | | 26 |
| Pac54 | | | | | 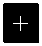 | 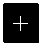 | | | | | | | | | | 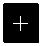 | | 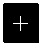 | | | | | | |
| Pac55 | | | | | 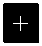 | 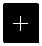 | | | | | 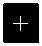 | | | | | 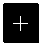 | | 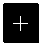 | | | | | | |
| Pac56 | | | | | | | | | | | | | | | | | | | 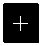 | | 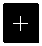 | | | 28 |
| Pac57 | | | | | | | | | | | | | | | | | | | 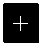 | | 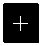 | | | 28 |
| Pac59 | | | | | | | | | | | | | | | | | | | 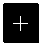 | | | | | 21 |
| Pac60 | | | | | | | | | | | | | | | | | | | 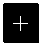 | | | | | 21 |
| Pac61 | | | | | | | | | | | | | | | | | | | | | | | | 22 |
| Pac62 | | | | | | | | | | | | | 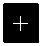 | 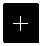 | | | | | 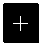 | | | | | 21 |
| Pac63 | | | | | | | | | | | | | 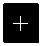 | 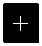 | | | | | 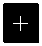 | | | | | 21 |
| Pac64 | | +? | | n.d | n.d | n.d | n.d | n.d | n.d. | n.d. | n.d. | | | | | | | | n.d. | n.d. | n.d. | n.d. | n.d. | 29 |
| Pac65 | | | | n.d | n.d | n.d | n.d | n.d | n.d. | n.d. | n.d. | 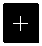 | | | 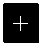 | | | | n.d. | n.d. | n.d. | n.d. | n.d. | 30 |
| Pac66 | | | | n.d | n.d | n.d | n.d | n.d | n.d. | n.d. | n.d. | 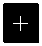 | | | | | | | n.d. | n.d. | n.d. | n.d. | n.d. | 32 |
| Pac67 | | +? | | n.d | n.d | n.d | n.d | n.d | n.d. | n.d. | n.d. | | | | | | | | n.d. | n.d. | n.d. | n.d. | n.d. | 29 |
| Pac68 | | | | n.d | n.d | n.d | n.d | n.d | n.d. | n.d. | n.d. | | | | | 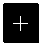 | | | n.d. | n.d. | n.d. | n.d. | n.d. | 33 |
| Pac69 | | | | n.d | n.d | n.d | n.d | n.d | n.d. | n.d. | n.d. | | | | | | | | n.d. | n.d. | n.d. | n.d. | n.d. | |
| Pac70 | | | | n.d | n.d | n.d | n.d | n.d | n.d. | n.d. | n.d. | | | | | 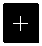 | | | n.d. | n.d. | n.d. | n.d. | n.d. | 34 |
| Pac71 | | 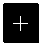 | 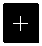 | n.d | n.d | n.d | n.d | n.d | n.d. | n.d. | n.d. | | | | | 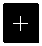 | | | 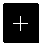 | n.d. | n.d. | n.d. | n.d. | 35 |
| Pac72 | | | | n.d | n.d | n.d | n.d | n.d | n.d. | n.d. | n.d. | | | | | | | | n.d. | n.d. | n.d. | n.d. | n.d. | 36 |
| Pac73 | | 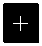 | | n.d | n.d | n.d | n.d | n.d | n.d. | n.d. | n.d. | | 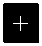 | | | 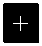 | | | 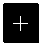 | n.d. | n.d. | n.d. | n.d. | 37 |
| Pac74 | 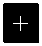 | | | | | | | | | | | | | | | | | 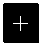 | | | | | | 39 |
| Pac75 | | 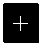 | 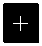 | n.d | n.d | n.d | n.d | n.d | n.d. | n.d. | n.d. | | | | | 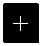 | | | 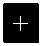 | n.d. | n.d. | n.d. | n.d. | 37 |
| Pac76 | | 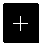 | 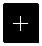 | n.d | n.d | n.d | n.d | n.d | n.d. | n.d. | n.d. | | | | | | | | 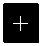 | n.d. | n.d. | n.d. | n.d. | 40 |
| Pac78 | | | | | | | | | | | | | | | | | | | | | | | | 42 |
| Pac80 | | | | | | | | | | | | | | | | | | | 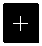 | | | | | 43 |
| Pac82 | | | | | | | | | | | | | | | | 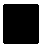 | | 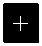 | | | | | | 44 |
| Pac83 | | | | | | | | | | | | | | | | | | | | | | | | 44 |
| Pac84 | | | | | | | | | | + | | | | | | | | 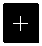 | | | | | | 45 |
| Pac85 | | | | | | | | | | | | | | | | | | 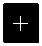 | | | | | | 46 |
| Pac90 | | | | | | | | | | | | | | | | | | | 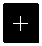 | | | | | 47 |
| Pac93 | | | | | | | | | | | | | | | | | | | | | | | | 49 |
| Pac98 | | | | | | | | | | | | | | | | | | | | | | | | 50 |
| Pac99 | | | | | | | | | | | | | | | | | | 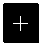 | | | | | | 51 |

Legend:
Pac: spot of Parachlamydia acanthamoebae proteome.
A: Patients tested positive with P. acanthamoebae.
B: Patients tested negative with any infection of Chlamydiales.
C: Patients tested positive with C. pneumoniae.
D: Patient tested positive for C. trachomatis and C. pneumoniae.
E: Patient tested positive for C. psittaci.
F: Immunized rabbits:
R1 and R2: rabbits immunized with P. acanthamoebae (Hall coccus strain).
R3: rabbit immunized with heat-inactivated Protochlamydia amoebophila (strain UWE25).
R4: rabbit immunized with Protochlamydia naegleriophila (strain knic).
R5: rabbit immunized with heat-inactivated Waddlia chondrophila (ATCC VR-1470).
R6: rabbit immunized with heat-inactivated Simkania negevensis (ATCC VR-1471).
All the organisms used in R1-R6 are Chlamydia-like and these sera are tested in western blots in order to evaluate the possible cross-reactions.
SQ: SEQ. ID. NO. (see attached official sequence listing).

With respect to A: these patients were tested positive by immunofluorescence using whole bacterial lysate. The positive outcome does not totally exclude cross-reactivity since human patients can have been infected by various organisms. Therefore, preferred peptides of the invention show reactivity to serum of at least one of the two immunized rabbits.

For the purpose of the present specification, the expression “to show reactivity” of a peptide when exposed to antibodies or to preferably diluted serum comprising antibodies refers to the presence of detectable binding of an antibody to the peptide under the conditions disclosed herein, for example in the ELISA described above. In case serum of a human patient is used, the dilution of serum is 1/64. In analogy, the indication that there is no reactivity refers to the fact that there is no detectable binding.

C. pneumonia and C. psittaci are acknowledged agents of pneumonia. For the purpose of sensitive diagnosis, it is thus preferable to selected peptides from Table 1, which do not exhibit cross-reactivity with patients suffering from C. pneumonia and C. psittaci infection.

1-24. (canceled)

25. A recombinant immunogenic peptide comprising an amino acid sequence selected from the group consisting of:
(a) amino acid sequences according to any one of SEQ. ID. NO.: 9, 2, 3, 6, 40, 1, 4, 5, 7, 8, 10-39, 41-57;
(b) amino acid sequences having at least 80% of sequence identity with any one of the amino acid sequences mentioned under (a); and/or
(c) fragments comprising 50 or more successive amino acids of an amino acid sequence mentioned under (a) and/or (b) above.

26. The peptide of claim 25, which shows no reactivity when contacted with the serum of an individual that is tested negative for infection by a microorganism belonging to the class of the Chlamydiales.

27. The peptide of claim 25, which shows no reactivity when contacted with the serum of an individual that is tested positive for infection by a microorganism selected from the genus Chlamydia but tested negative for an infection by a microorganism of the Parachlamydiaceae.

28. The peptide of claim 25, which comprises an amino acid sequence selected from the group consisting of:
(a) amino acid sequences according to any one of SEQ. ID. NO.: 9, 2, 3, 6, 7, 14, 17, 18, 26, 28, 35, 37, 40, 43-47, 51;
(b) amino acid sequences having at least 80% of sequence identity with any one of the sequences mentioned under (a); and/or
(c) fragments comprising 50 or more successive amino acids of an amino acid sequence mentioned under (a) or (b) above.

29. The peptide of claim 25, which comprises an amino acid sequence selected from the group consisting of:
(a) amino acid sequences according to any one of SEQ ID NO: 9, 2, 3, 6, 26, 28, 40;
(b) amino acid sequences having at least 80% of sequence identity with any one of the sequences mentioned under (a); and/or
(c) fragments comprising 50 or more successive amino acids of an amino acid sequence mentioned under (a) or (b) above.

30. The peptide of claim 25, which is purified and/or isolated.

31. A pharmaceutical composition comprising the peptide of claim 25.

32. A pharmaceutical composition comprising the peptide of claim 25, and comprising at least one further, different peptide comprising an amino acid sequence selected from the group consisting of:
(a) amino acid sequences according to any one of SEQ. ID. NO.: 9, 2, 3, 6, 40, 1, 4, 5, 7, 8, 10-39, 41-57;
(b) amino acid sequences having at least 80% of sequence identity with any one of the amino acid sequences mentioned under (a); and/or
(c) fragments comprising 50 or more successive amino acids of an amino acid sequence mentioned under (a) and/or (b) above.

33. A vaccine comprising the immunogenic peptide of claim 25.

34. A diagnosis test kit comprising the peptide of claim 25.

35. A method of diagnosing an infection of an individual by a Parachlamydiaceae microorganism, the method comprising the steps of:
contacting the serum and/or a blood sample of said individual with the peptide of claim 25;
determining the presence or absence in said sample of an antibody specifically binding to at least one of said purified, recombinant and immunogenic peptide; and
diagnosing an infection if an antibody is detected in the previous step.

36. A method of diagnosing infection by an intracellular Chlamydia-like microorganism, the method comprising the steps of:
exposing tissue to an antibody specifically binding to the peptide of claim 25;
detecting if said antibody binds to said tissue; and
diagnosing, if there is such binding that there is an infection by said Chlamydia-like microorganism.

37. The method of claim 36, wherein the tissue is a tissue sample taken from an individual.

38. The method of claim 36, wherein the step of detecting if the antibody binds to the tissue comprises the step of detecting an antibody-antigen interaction, wherein the antigen is the immunogenic peptide.

39. An antibody specifically binding to the peptide of claim 25.

40. A method for producing an antibody, the method comprising the steps of:
(1) exposing a mammal to the peptide of claims 25; and
(2) harvesting the antibody from serum of the mammal.

41. A diagnosis test kit comprising at least one purified, recombinant immunogenic peptide comprising an amino acid sequence selected from the group consisting of:
(a) amino acid sequences according to any one of SEQ. ID. NO.: 9, 2, 3, 6, 40, 1, 4, 5, 7, 8, 10-39, 41-57;
(b) amino acid sequences having at least 80% of sequence identity with any one of the amino acid sequences mentioned under (a); and/or
(c) fragments comprising 50 or more successive amino acids of the amino acid sequences mentioned under (a) or (b) above.

42. The diagnosis test kit of claim 41, which detects the presence of an antibody specifically binding to a peptide expressed by an intracellular bacterial strain of the family Parachlamydiaceae.

43. The diagnosis test kit of claim 41, which is a serological test.

44. The diagnosis test kit of claim 41, which is an ELISA (Enzyme-Linked Immunosorbent Assay.

45. The diagnosis test kit of claim 41, which comprises at least one further, different peptide comprising an amino acid sequence selected from the group consisting of:
(a) amino acid sequences according to any one of SEQ. ID. NO.: 9, 2, 3, 6, 40, 1, 4, 5, 7, 8, 10-39, 41-57;
(b) amino acid sequences having at least 80% of sequence identity with any one of the amino acid sequences mentioned under (a); and/or
(c) fragments comprising 50 or more successive amino acids of an amino acid sequence mentioned under (a) and/or (b) above.

46. An isolated, recombinant immunogenic peptide of an intracellular microorganism of the Parachlamydiaceae, wherein the peptide shows no reactivity when contacted with the serum of an individual that is tested negative for infection by a microorganism belonging to the class of the Chlamydiales, and wherein the peptide shows no reactivity when contacted with the serum of an individual that is tested positive for infection by a microorganism selected from the genus Chlamydia but tested negative for an infection by a microorganism of the Parachlamydiaceae.


SEQUENCE LISTING (71 sequences)

SEQ ID NO: 1 - protein, 493 aa, Parachlamydia acanthamoebae
KSIGANSAMNFKSSFTQFEKIFCSLLLTTFSLYVHPAFSATTTEIQQQHESPKIMALDFVDVAKKATPAVVSIRVKIAPSSPFQKSERDETDLFNENFWQQFFGLPKSNEKKSSQDQVGQASGFIVSDNGYILTNNHVITDAKEITAMLVDGREFPAKVVGKDKNTDIAVLKIEAESLPYLKLADSDELQPGQWAIAIGNPLGLQASLTVGVISATGRDNLDIATIEDFIQTDAAINRGNSGGPLLDMKGEVVGINTAIVSNQGGYMGIGFAIPSNIAQNIMDQLISSGSATRGFIGVTLQKIDQNLAQAFGLTKMEGALISDISKGSPAEKAGLRQGDIVLKYDNHPVAHISALRKAVSFMKPGTKLNLTILREGKTLEIPIDVGTFPEHPSQLIAADNKIGLEVENITPETAQKLGISGLKGGLLITQVRPGSPAYIAGIRPGAILVAVNQNTVTTVAEFQQILKDSDPSKPVLLLIKQGGFTRFVSIKVD

SEQ ID NO: 2 - protein, 264 aa, Parachlamydia acanthamoebae
IRRNFFMAISLYSNQQPFAKLIEEASKQLTGHSELRLSSGLKVRKAGYGFIEWLRSIFGADSTRGYKKTAAKLEALALKRSAEIPFEGDLRDQFNEHYKNILGKVLHITEKDDGSLEVDAKLTKNVKKTEAIKKRFTDSLDKVKKIEVTEEAKEIQRKQLNILNELEDLKADKAAKAQEITGKHEEISQQQKAIEDLQALEDKDEDAIAAAKETLKELSDQLSRLEEENDALAISQKTKEEERTKNHAALIAFQVKKKPATSNT

SEQ ID NO: 3 - protein, 696 aa, Parachlamydia acanthamoebae
RMARGEKNQLKNIRNIGIMAHIDAGKTTTTERILYYSGRVHRMGEVHEGAATMDWMEQEQERGITITSAATTVFWKDAKINIIDTPGHVDFTIEVERSLRVLDGAVAVFCSVSGVEPQSETVWRQADRYGVPRIAFVNKMDRVGADFADAVRTMREKLHANAIPVHCPIGAEADFKGMVDLVSMRALFFHDETLGAQWEETDIPEDLLEKCKQMRTELLDELATIDDSDDEFMTKVLEDPDALTVDEINAVIRKGVCANKFNPVLCGSAFKNKGIQQLLDAVVSWMPSPLDRGAIKAHDLNTDEEIMLQPSDDAPFSALAFKIMTDPYVGRLTFIRIYSGMLTKGMNLINSTKDSKERISRLLEMHANKREERDEFYTGDIGACIGLKKASTGDTLCEAERPFVLEKMEFPEPVISMAIEPKSKADREKLSGALSALSEEDPTFRVTTNEETGQTIIAGMGELHLEILHDRMKREFGVEANVGKPQVSYKETITIPGASQTKFVKQSGGRGQYAHVELEVQPNEKGKGNEVVSKIVGGVIPREYIPAVIAGVNEGLATGVLAGYNLVDVKVAIVFGSYHDVDSSEMAFKICGSMAIKDAARKCKPIILEPIMKVDVTTPEASLGDVIGDLNRRRGKILGQENHKGSVIVSAEVPLSEMFGYSTQLRSLSSGRATYTMEPSHFEKVPAKIQEEITKK

SEQ ID NO: 4 - protein, 552 aa, Parachlamydia acanthamoebae
ETTTKRSLRSMIMAAKNIKFKEDARQNILKGVRTLASAVKVTLGPKGRNVIIDKAYGAPHITKDGVTVAKEIELEDKHENMGAQMVKEVASKTADKAGDGTTTATVLAEAIFSEGLRNVAAGANPMDLKRGMEKAVKTIAKELEALSKPIKNQHEIAQVATISANNDAEIGQIIADAMEKVGKDGTITVEEAKGFETTLDVVKGMNFDRGYLSAYFMTNPETQECILENPYILIYEKKISVVKELIPLLQTVAESGRPLLIIAEDVEGEALATLVVNRLRAGLKVCAVKAPGFGDRRKAILEDLAILTGAQVISEELGMKLEQATIEMLGKAKKVIVKKDDTTLIEGQGDKERLRDRVAQIKRQIEETESDYDREKLQERLAKLAGGVGVIRVGAATEVEMKEKKDRVDDAQHATAAAIEEGILPGGGVAFIRCIPALNRLATTLEGDEKTGVLIIARALSAPLRQIAENAGQEGSIILQEVESRPVTEGYNALTGEYVDMFESGILDPKKVARCALENAVSIAALLLTTEATVVEIPEEKAAPAMPAGMDY

SEQ ID NO: 5 - protein, 561 aa, parachlamydia acanthamoebae
LMSKLLQFNEEALKSISKGLKTLAKAVKVTLGPKGRNVVINRGFGSPLSTKDGVTVAKEVSLKDKFENIGAQLVKEVASKTSDIAGDGTTTAIVLAEAIYSAGLKSVTAGANPMSLKKGIERAVEVLNKELTALASPVNTSQEIQQIACISANNDPEIGEIIAKAMEKVGKDGIITVAEAKGIDTTLDVVEGMQFDKGYVSPYFITNPENMSVELQNALVLVTDKKLSSAKDLIPILEKTMEKGARPILIIAEDIDGEALATLVVNKLKAGLPVCAVKAPGFGDRRKALLQDIAILTGATVVSEEVGLQIEEVDVSVLGRAKTIKITKEETTIIDGMGDAGLIQDRINQIKAEIANPSTSNYDREKLEERLAKMVGGVAIINIGAATETELKEKKARVEDALHATRAAVAEGIVPGGGVALLRAVKSLDNLKASGDEATGIAIIKSAAFAPATAIANNCGKQGNLIAEKIFEAQGAHGYNGLTDEFCDLVQAGVIDPVRVTKSALTNAASIAALLLTTAAVITDKPEPKSKQPDMHAMGGMGGMGGMGGMGMGGMGGMGMM

SEQ ID NO: 6 - protein, 342 aa, Parachlamydia acanthamoebae
ILKIERLRKRMKIGYLGMGAWGFCLASLLAAKGYELTCWTTKQALADRLNQTREHPSLPGHIAKGNLHFTTDLKAALIQSELLIESVTSAGLRPVLEKVKAVGPLNIPLIMTSKGIEQGSGLILPDVAIQTLGPQVKSQVGFLSGPGFAEEVIRNLPTSVVGSAYDPNLLMTVCQIFTTPTFRVYPNADIHGVAFGGALKNIIAIACGICEGLGLGNSSKAALMTRGLHEIRKLAVAQGCKAETLNGLSGMGDLFLTCSSFISRNFRFGHLMTQGLTPKEAQDKIGMVVEGAYTCVSALQLSEKLNVSMPITKIIHEIIYKDMKPQIAVNALMERAIKEEHL

SEQ ID NO: 7 - protein, 607 aa, Parachlamydia acanthamoebae
KEQEIKGDHMNKKKGKIIGIDLGTTNSCVAVMEGGVPKVIASAEGSRTTPSVVAFKGNERPEEIAAQILIKMKETAEAYLGEKVTEAVITVPAYFNDSQRQSTKDAGRIAGLDVKRIIPEPTAAALAYGLDKEKTEKKIAVFDLGGGTFDISILEIGEGVFEVLATNGDTHLGGDDFDHAILNWMLETFKQETGIDLHNDKMALQRLRDAAEKAKIELSGTQSTEINQPFITMDATGPKHLSLNLTRAKLESLTAELIDRTREPCIKALKDSGLSKDDIGEVILVGGMTRMPAVQEVVKSIFGKEGHKGVNPDEVVAVGAAIQGGVLAGDVKDVLLLDVTPLTLGIETMGGVMTPLVERNTTIPTQKKQVFSTAADNQPAVTIRVLQGERKMANDNKEIGRFDLADIPPAPRGVPQIEVAFDIDADGILHVSAKDNSSGKEQKIRIEAQSGLREEDIQNMLKDAELHSEEDKKRKEEVEIRNEADSQAFRASKALDEYKDKLPAEIVSEVQGKIDAVKKALEGTDSARIKSAKEDLEKSMQHIGEAMAKAGAAGGAHASAAAHEGQAHQQSSSFEGGQSHGGHHHEHSKDDDQIEEAEVEIIDDKDK

SEQ ID NO: 8 - protein, 624 aa, Parachlamydia acanthamoebae
KIRSMIMVTKTLEIHSENILPIIKRWLYSDKDIFIRELVSNACDAIHKVKILQDKGELSASSDDPFRIEIHIDKENKTLTFSDNGIGMDAEEVQKYIAQIAFSSAEEFMEKYKSHNETDQFIGHFGLGFYSAYMVASKVEINTLSYKEGAEPVRWICDGSAQYELEVGTRTKRGTEVILYVDKDSEEFLDPARIRQILNHYCSFLPYPVYLEDSHINHEEPLWIKSPSECTSEDYLRFYRHLYPMDPDPLFWVHLNVDYPFHLKGILYFPKLNRDFDISKNTVKLFCNRVFVSDNCKDVIPNYLMALRGVIDSPDIPLNVSRSYLQMDRTVRQLGNHISKKVADSLATLYRTERERFLECWSDVGQVVKLGILEDEKFYDKAKSFLVWKNVNGEWITVEEYLERNESKIKDKVFYTIDGEHLSHFAEVYHKQGIEILVANSPFDPYLIQFLERKLTPVVFKRVDSDIDESILDKEKEKTLLDAEGRTEAGKIADFVRSKLENENVEVEAKSLAAESVPGVVVIDEQQRRMRDYMRSLDPKDASRVKGLMDKRKLVINTNSPFVSLVQKLDQIQPELTGDLVKQIYEMALLSQKEMDPAELKEFLERNARLLEKLTEKLIDAKQQ

SEQ ID NO: 9 - protein, 520 aa, Parachlamydia acathamoebae
FNKIRHFMTYNNNINVLQAKSFGKDLIAIEKDGNTLKSVGFLGRFIRRLKKMIGKNSYEDCKITKIASTMKNMVDEHPSDEVGKKIVQDKFKEILGLKKIKPENKDLVQQIFFEVFPKESLKHDLLEKGFGALDRRSFDEIKELIDLLTDRELNEVLLKEDSLNPDNKDALPLISTAIMDISDPAKALSIVQELHARGIELNSKAVEHGEASTLYELALRKNYREVAEFIAAELGFPPNANVSTDAELQRINLMLDRLNDAKTEAEEERIFKDINDLTEEGKLNNVFSLTTKDGSVENHTLLTAIYTTLDDDEKKWKYVSYLIEKGADLNMKIGEPKESIGALLCRDFIDEFASFSPTQISNNVDIFGEYHADGEAGLKFELLEKAIHDQGDRKWDIISRLFDRNVDLNREIGPEDSKEKIGHLVLKDLVSEIDNIPTAQILSMIQDLKNYNLINLPVQIQDEKNILITTLLVEANKKLNKDGKSIVVKTLLEKGADPAFRPIIVSPLTGVNTQRSDQIP

SEQ ID NO: 10 - protein, 121 aa, Parachlamydia acanthamoebae
KIQIVGDDIFVTNPKFLQKGFEEKIANSILVKVNQIGTLTETLETIRLAQTHAYSAVISHRSGETEDSIIADICVATNSGQIKTGSLCRTDRVAKYNRLLSIEAELGSIARYADSHSAKKI

SEQ ID NO: 11 - protein, 444 aa, Parachlamydia acanthamoebae
IASPLKSSVKEVLVKKTFVLDTNVILHDPEAIIKFPRNRVVIPVAVLEELDKMKRLPNDLGKNSRAFFRFLDSLASEGKGDLHNGISLSNDSDVRIALEIKKDYKGDFSLSTTDNKIIMAAYLLFERSENVVFVSKDFAARIKAEAIGLEAEDYENLKYAYQTMYRGNRRVEVTKHDVDSFFKDGFIKLPDLDCHPNEYLVMTSPENSSAVGKYDPVRKQVEPLLKSVNMWGIKPRNVEQRCAVDLLLRDDIKLVTLLGPAGTGKTLLALACGLRKVFDEGIYSRILVSRPVIPLGRDIGYLPGTKEEKLFHWMQPIYDNLEFLCESPSGQTTDTLRWVTESKKVEMEAVTYIRGRSLPKMYIIVDEAQNLTPHEVKTIISRAGEGTKVILTGDPTQIDHPYLDKDSNGLTYAVGRFADQKIYGNMFLEKTERSELAALAAEIL

SEQ ID NO: 12 - protein, 464 aa, Prachlamydia acanthamoebae
EICNFHKAPTRKNMRKCLNFFSAIREEKVQKGTKISSEEENAQEKRKALLVSAFNGNDQRAICEEHLDELELLAETYGVEIVAKVPCLIRKVDAATFVTQGKLEELITLSKECAADLIIFDDEISPSQQRNLEKAFQLTVMDRTEVILEVFAQRAKTKEARLQIEYARVKYQAPRLKRLWSHLSRQHGSGGAGGGAYLKGEGEKQIEIDRRLIKKRLEQLQSEIQEVKEHREIQRVARTRSAIPVFALVGYTNAGKSTLLNALTDADVFVEDKLFATLDTTTRKFPLSTGQEVLVTDTVGFIRKLPHLLVAAFKSTLEEAIQADILLHVIDATHPMALEQARTTIQVLKELGVGDKPIITVLNKIDQAEQNELTTRLRLEYPRTVPISALHRRGFEELEEMMLRELSQQRKTLNLRIPQSEYALVSEIRRSGNVISQDYEENDVLIEVDLPVALANKLKNYVIS

SEQ ID NO: 13 - protein, 401 aa, Parachlamydia acanthamoebae
PNGGNQMAAKESFKRSKPHVNIGTIGHVDHGKTTLTAAITKVLAEKGGAIFRSYDSIDKTPEERARGITINSTHVEYETENRHYAHVDCPGHADYVKNMITGAAQMDGAILVVAATDGAMPQTREHILLAHQMQVPAIVVFLNKVDMLSEGDEELLDLVEMEIHELLEAKGYKDAPVIRGSGLRALEGDPKYVEAIHKLMDTVDAFIPEPAREIDKAFLMPIEDVFSISGRGTVATGRVERGMVKLGDKLQLVGLGDTRDVVVTGLEMFNKTLDEARAGENVGILLRGVDKNQIQRGMVLAAPGACTPHTKFKGPVYIQTKEEGGRHKPFFTGYRPQLYIRTTDVTGTVELPKDVEMVMPGDNVEMIIELIQPVAVEKGMRFAIREGGRTIGAGTVSEIIK

SEQ ID NO: 14 - protein, 280 aa, Parachlamydia acanthamoebae
TMSTPVTAAMIKELRERTGIGMGACKKALEEANGDMELAITNLRKSGAASAVKKEGRTTNEGMIGEAQNDKAIALVEVNAETDFVVKNERFQQFVQAIAKEIADTNPVSLEAFLQQKYSQDSNMTIDDLRSSLVQAIGENIQIKRLKVLPKKANTSVGVYSHLGGKMVTVVEITGSNEQEALAKDIAMHVAAAAPEYVSPEQVPSEVLEKEKEIARSQVVGKPDFVADKIIAGKLNYFYDTACLVCQKYIKDDSVKIEDLVKQKGKDLQLTSFERWTVSQ

SEQ ID NO: 15 - protein, 274 aa, Parachlamydia acanthamoebae
STRLILFPIRRRNLFMIKKSRIWALVALSSLFLQAGALSADQTATQQESKPAQAAPQEIDIMKLSEAFGNFIGRNLNAPGIKFDLESIIKGMRDGAANKPSPLTDQEYETMMTQLQAKAYTELASENLKAANQFLKDNLKTANLVEIEPGKLQYVIVEAGHEPTVTEHASPLVKYTGKYIDGSVFGSSDDTGGPITVPLDQTIPGFSKGILGMKEGEKRKLFVHPDLGYGTSGHLSPNSLLIFDVEVIKSDNSAEDKSKPLPDFSDENSDKNSR

SEQ ID NO: 16 - protein, 598 aa, Parachlamydia acanthamoebae
KHIKKLLKQVNKNIESEEFDMPDPIKGFAQVAYALNKAADYVKQSTTALGHQISNVLGQKTVPVEKKIQDQTPKMETQAPTRKVRFAVSEPTKETLENQTVKKTMTAFQSFFSQIKQHFESSYELIKTKDELKQEVTQILNENQSKPFNELTAHEQGIIKLYISSADKSILDTLRTLADEKNPKTLDLLLSLPPEYQSKFFAKLSPDTGVKMRDFLIQQTKSSRQDDVFQLLNSTPLPFYKSVLNSLLTPNYTEKMTNPTTSQRDLVSHTIRDSIINLCHADTESLVKNQNLLLNVPANHLTSMLKEVWEENPEAALHTVQVLSSRIDESPETAKGISQFVQNLNEKQQNTLFEMTLEHPPRGISQILLALPSSVSVSMAEMMINSGALSDNDLADIASHLITNEVKTLDSLQTFLVKDNVAMKFVSMLQRKENEKSLNYIWGHIPNNFELTHSNLETKEIIVPNPENFLNIHQTILKDITHHLETNPPTPAMKKVYQHLQNEIDRKFPSANHENGKIAVLGLYLIKMQNAMLVNPQQFDDSFVLIDKQAPNAIEIAKILTKFALGEKFEASSNYAFMNPFFEDTTQLRNQMFQGLTQ

SEQ ID NO: 17 - protein, 399 aa, Parachlamydia acanthamoebae
HMNINSTPPDVTTQGYNFDPSIFAKFEKAMSQAEDSSEGGAPPLTGEQIKKHVVDSKGKAVTPVGISDSQSNQAVSATASSASPGVALDAQGNKVAVVSTVMQDVIEEASTAMDKFSSAYVSGTNQSLILNNPADTPTLVAPTGTGALPTGSKGHGNPFLTASFVVALIEVMAELIKVQSQQRLVETKLDIMSNQWTIDLAKRMASDIMQSAKTEAMMHYMLAASAGVQLGMAVANGAMGALAMKKSIGDYNKQTKALQTKFDELNAPAKSRAPGSDAKVGTEDQLMKAKFKLDEHRANKWSNISTSFRMRMFPMDTAKDITDSTSKMYENIVQGIMKVKKAEYDADIKLAEGYQEIARKIGADASASLKDIEDKVKSIAEAVQQMITKLYSAFNYRIH

SEQ ID NO: 18 - protein, 232 aa, Parachlamydia acanthamoebae
CLKNEVMMKKIWLVAISLMMGVAQMAMGDETASSTKKYEVRDFDHLLGKVKGLNDDLLKMHFKLYQGYVNNTNTLLQKIGELDQTGKSQSPEFAGFKHMLGWEFDGMLLHEYYFENLGGQTHQLKQDDPLFLKMVQDFGGYDQWKSDFQATGAIRGIGWVITYVDPKQGRLVNTWINEHDVGHLSGGKPLLVMDVFEHAYITQFGLDRAKYIQVFFDNIDWNAVSQRYKKTL

SEQ ID NO: 19 - protein, 235 aa, Parachlyamydia acanthamoebae
LKLILKSKNLKSRKQIHMQRSLGGRNIRRSLSIISELSMRTSHKSLTFLLRRMAMKKLSLILFFALCAFVFNSGYAQCGPSGCGVPASGGFEGGSSFAAPQGGEGGEAGACGEYAVGDCYCLFVKYEPRYCNKYRCEWDTQSYQVQRCRQVPETYQKTCCRYVPQYYQVDCVRYVPQYYCTTETRQVPRYVCDRECKYVPKYYYKRICNRNPVPPAPAPTTVTTPAPASCCGSQY

SEQ ID NO: 20 - protein, 224 aa, Parachlamydia acanthamoebae
VYQSLSKGFSMSTKVEQKGASSQRLKDELKELKSKKGKNQESSRIHEIAKQLAAEKEWSSAIEAIQLITDEKERNILISDTIEGYLLPAKEIDQAKKFAKLLTPTPEMEPFVWIRIALASKDPEQALKLAKQLPSPISRNFAFIHIMEYYLSNKEKSKIDELRKQMLENLKTIYDHKSRSFILRELAISLFYAYGEKEQAKEIAKLIPDEDMRSKVLKKVEATK

SEQ ID NO: 21 - protein, 329 aa, Parachlamydia acanthamoebae
TNIFKQKKYVSYLTIALGLSVAPSLSAFSYDNSSSSNPSSNYSNPSSNPGSARDSYSGSSSKDSYSNPGSSGSRDSNSGSSGSRDSSSNPGSSSSRDSDSSPGSSSSGGSSSNPGSSSSGSNDSFSNYSSQRDVNPNSSSYYNPSGSSYSNAKDPYSGSSSATYSETSGDQELARRIREKISSGWFSRGYDRVSVQVNNGAVIIQGVVKSQADKDKVEKEIRDIDGVKSLTSHVNVEESHARGHEERQFLQDRAATSEDQQLNKKIRDNVSKGWLWDSYKDVSLNTSNGVVTLDGTVGSESDKQDLINEVQKVEGVKSVKSNLRIESRY

SEQ ID NO: 22 - protein, 338 aa, Parachlamydia acanthamoebae
WSVSMGPNLSVTSDDADRYRSEGPDRKLLKDTFKVVHKDDAKEDSQKKRKNVADKGKFTKALDAANLKAEQEEEAQSKPLSPSLFDLARGRKGAGDKGIEDKGAKEQAVQNQGEEEFEQSRSQIAQRPYFVPHAEKKPEVKMEDVSINQNDTEDFGAGVDSKKKNGQNPVQELPNQPLVNPMVVKTELVSIYTPTPPIVQPVNMIDRIKEIVDQIGQIDLYSIKEGGKTETAITLNGINKMFDGARVVVSSYDTARNEFNITFENLTQQAKNLLDLPKNQEALMQTLAQRGEWIVHQVTTTTIMENRPYLDDTQLAKDDHQRDEDESQGQGRQKRQKG

SEQ ID NO: 23 - protein, 590 aa, Parachlamydia acanthamoebae
MSNKFKRAWEESNIVDDVMFDEKDAQAFKAMLDQKGGDVSIEEPTAMIPGTVLKGRIVEITKDHVVVDVGLKSEGLVPMEEFSDPSQIVLDGEVEVLLDQAEDDNGQIVLSREKAERLRQWEYILQHCEEGSIVKGRVLRKVKGGLMVDIGMEAFLPGSQIDNKRIKNLDDYIDKTYEFKILKINIERKNVVVSRRELLEAERVSKKAEVLERIQEGDIREGTVKNITDFGVFLDLDGIDGLLHITDMTWKRIKHPSEMVQLGDKLKVKILSIDKEKGRVALGLKQKEPNPWDQIEQKYPPGTRVKGKIVNLLPYGAFIEIEPGIEGLIHVSEMSWVKNVTDPSDVVKKGDEVEAIVLSVQKDEGKISLGIKQTEHNPWDDVQNKYQVGTNVKAEIKSLTNYGAFVELEPGVEGLIHISDLSWIKKVSHPSEVLRKGDVVDAVILSVDKDSKKITLGVKQLSDNPWESIEKTMPVGTLVKGVVSKITAFGAFVELESGIEGLIHVTELSDKAFGKVEDVVAKGDEVIAKVIKLDPEYKKIALSIKEYLIDQNQCNHDDIVVAPSKRKKKDAEEGRKDDDENTESDESDES

SEQ ID NO: 24 - protein, 440 aa, Parachlamydia acanthamoebae
DVLSNKLPKYEVMVSNNQTEVSNEHVKISLKKEDGCLVKLDVHVTPQATQAAYQEAIKAISKEVSMPGFRKGKVPSAMILQNFHPHVEKEWRDILLNRALREFMDITRIYPFTKESVQKALVKNVSLEAGADLLIDFESAPEIPEVDPKSLELKSAQKKDITEADIDKALENVRIHHADWNVVTDRPVQEGDTVVVDACSVENPEQVYYADASFKVDEQNMAEWFKTAIVGHKTGESVEAQAADQEPPVHVRITIKEIKTAILPELNDEFAKKSGAESLEDMRKKIEDQLNKNAEEESKFVLREQISPILLEKYPFEVPASIVKQELKRIITQKTEELKETGLSSSEASKKIEEMREQIEKECTDSIRLIFLENELIQKFNVQVSDEEVVRELFAQIYKYSGSLTDARGLLNSPEMHNRVRQLLMSKKIKDFLVEQAQKN

SEQ ID NO: 25 - protein, 542 aa, Parachlamydia acanthamoebae
IIYEKLKEKNNMSSTPKQIIFEEEARELLLSGIKKLADVVAFTLGPKGRNVGLEKSWGTPTITNDGSSIVKEINVQCVYENMGVSMGKEVVQKIKEKCGDGTTTGTLLLSALVENGVKLIASGASPIGIKRGIDKAVEAIVKAISTSAIPVKSSQEVRHIATASASGNEEIGNLIAEAMEKVGKSGVITIEEAKGIETTIEIVEGMQFDRGYISSYFCTNTDKMIADMANPQILLVDRKIGSIHELLPVLQATASAGKELLIVAEDIEGDALSTLVVNKLRGTLKVVAVKSPGFGDRRKAMLQDLAVLTGATVISEDAGMSLKEIPASALGSAEKIIVSKENTVIIHGSGSQEDIDARVKQIENEIEITKSSYDKEKLEERKAKLSGGVAVIRVGAATEPELKQKKQVFEDSLNSTKAALEEGIVPGGGVALLRAKSAIGQLKLVGDEAMGATIVEKACETPLKQIVSNAGLDGSVILAEVLKAPYNFGYNVVSQKLEDLVASGVIDPAKVVKNALIYAASVAGIVLISEALIADAPEDDEE

SEQ ID NO: 26 - protein, 324 aa, Parachlamydia acanthamoebae
EMKAIVVSQYGDPEVLSYQDADIGKPGKGQVLVRLAYAGVNFVEIYQRRGTYPRTLPYIPGSEASGIIEAVGEGVEDFKVGDHVAYVHQPGAYAEASIINAKSLIPLPSSFSLEQGAAFPLQGMTAHYLLHEFRKPKQGDVVLIHAAAGGMGLLLVQWARHLGARVIGTVSTEEKAKAARQAGATDVILYNEKDFAVETKRLTDGHGADLIIDGVGKTTFKGNLEAAALRGHIVIYGAASGPAEPIPPNALMVRSLTVSGGSLPNYLLTRDEMLQRAHSVIKGIQEGWLKLTIDKVIPLDQAAEAHRLLENRQTIGKIVLKIKA

SEQ ID NO: 27 - protein, 208 aa, Parachlamydia acanthamoebae
GKMAFNFLKNSFEKMKNALTHAGSLLGNKIRALFQGPIDDETWGKLEQLLYEADLGVQTAIELTEKVKKLSQQNSSLKGDELLSSVRTEILQVLNQHASPLREISSSSEPLVILIVGVNGNGKTTSTAKLAKFFQSQGKKVLVAAADTFRAAAVEQLDVWAQKLGIDIVKGAPKSDPAAVVFDALSAAKARQAEVVIVDTAGRLQTKN

SEQ ID NO: 28 - protein, 252 aa, Parachlamydia acanthamoebae
NRRKQFDKIDSIVLAYLYWPNSIGLEKEVFMNRDVLETNWVQVREILREKFSNLSEDDIRQINGRYDQLVAKLQQKYGYSKEEAEDRIRSWNFDRMAAGPRDAIRDEKLRREDANTIRREDTARREDRVRREEDNSTLFKWLLGLGIPLLLLATYFLSSGTRTPEVTRTPTATQEQFVVETPADRAISSSLRNAFLSQPNLASELQTVQITTHNGVVTLSGSVSSREISDFMTTSAQNASGVNQVINNLQIR

SEQ ID NO: 29 - protein, 153 aa, Parachlamydia acanthamoebae
FFLTPILKYLRRVIMKKVIYLLPALGMLFTACEKKETPTGPTPPNPSSYYTADADSALDATSTKSKDASITDTHPGDQADSYADRPWLRKIRQAIAEDKDLESQAGNIKIVIIKGTINLKGTVPNEKAKADLVKKVKQVVGDRKVEDQTEVSK

SEQ ID NO: 30 - protein, 235 aa, Parachlamydia acanthamoebae
NQFVIPTILELKHCSCAPSIKIMNELVFKDTLFIHIKSSRDLDLFLLKKIEKKCRINLWSNFKYLQLREVFMIKTRHIFPLFAIAASCVLFNPSTLRAANISSVQAQADEQNFYKQIEDLIAKDQKHLQEVQDRAYSRTEAPMWATKVELEQAIIMLDVKKVLYANFKNTPAIQSPLVREKLLSLMRKDNITMSDLAELQSLTEREKQHLRDESAQIQSQQTQPQQPPVPAGSAQ

SEQ ID NO: 31 - protein, 171 aa, Parachlamydia acanthamoebae
LILLNLYKEIKMKSKLLILPALSIMLVGCEQHVTNPPPPKSPTQQNSSILDAAPSETNTNRNLQDRRASDDADNTSRNVRDRIEGALTPFDQSESVGDRITLQSIRKALVENKILSTYAKNIKVITANGVVTLRGPVNSAAEKEAIERIVSLIPGVTKVDNQLEVLQNAEK

SEQ ID NO: 32 - protein, 182 aa, Parachlamydia acanthamoebae
VQSNKLKEKMEAALEHLKNDLNSIRTGQANPGMLDGITVEVYGSQMRLRDIATINSPEARQLLITPFDAGTAGAIGKSIERANLGFMPIVDGNAVRIKIPPMDESLRKEMAKLCHKKQEEAKVAIRNHRRDANDLVRKQKAAGELPEDQMKKQEKEIQELTDKYCKLADEIAAKKEKEISTI

SEQ ID NO: 33 - protein, 95 aa, Parachlamydia acanthamoebae
QEVIMVNKDTLQGQWKQLQGKIKEKWGKLTDDELTKINGRREMLIGKLQEKYGMAKDRAEKEVQNLEEAFRHSSESSEHEHEEKEHAHGSSKRRF

SEQ ID NO: 34 - protein, 133 aa, Parachlamydia acanthamoebae
IKSNRLVYKEKRMALKKTEVAKFKKRLEEMRDHLTRMLKGSTEEVKKPDEATGYSQHQADQGTDDFDRTINLEVTTREYAILRQIDRALEKIHDNTYGICDVTGEEIPLARLEAVPYASMTVKAQEKLEKGLI

SEQ ID NO: 35 - protein, 146 aa, Parachlamydia acanthamoebae
ESAKKACKKIGRAIYLLISISLQALLARCKPKLRRNPMAQTEELKATQAKNTLKPLGDRVLVRRLEAEEKLKGGIILPDTAKKKQEQAEVVAIGTGKKDKDGNLIPPPVKIGDIVLMEKYSGQEVTLGDQEYVIVRGDDLIAIIQN

SEQ ID NO: 36 - protein, 147 aa, Parachlamydia acanthamoebae
IMARDLLPDLISDRPFTQLANLWPNLFAGNVLSDDFSIKGVRIYEENNQLHVEVPVPGLSLDDIEVSLNKGVLWIKGELKEEEKDKKRNFYRFSKRSYSSSIPLPAQIDEKQEPQAVYEDGILKVSLQLAKHAETKKIKVKSGNKKK

SEQ ID NO: 37 - protein, 107 aa, Parachlamydia acanthamoebae
ELKTALEEKWGVKAAAAAAVMAVAPAAAAPAAAESTDFQITLEKVTDDKKISAIKVVREITGLGLKEAKELVESTPKVIKETAPKAEAEEIKKKVEAAGCKVTLKGL

SEQ ID NO: 38 - protein, 129 aa, Parachlamydia acanthamoebae
PKVVSMNCIVKSKRHVTLIEMMIVMFLIALITGVVAYNYRGALDEGKVFKTKAGIERLETILNLQVSENPALLNDIGNNWQGIVAKSPLVQNPAALIRDGWGEEYKVTVDNGVIHVRSEKLEQYQKSSK

SEQ ID NO: 39 - protein, 175 aa, Parachlamydia acanthamoebae
VWGGDMTQNAFYRLFLKEISDLYSAENQLVEALPKMAEAATTPELKEALERHLSETKKHVARLDSIFSMLNENSFKETCEAMEGLIYEANEIIQNEAPSVVKDAALIGATQRIEHYEIAGYGVAKTFATQLELYDIAELLKESLEEEGNADKHLTVIAEGGFFTTGINKRAKNIS

SEQ ID NO: 40 - protein, 98 aa, Parachlamydia acantamoebae
IMANIQPLADRVLVKRTKASTSKGGILLPDSAQEKPKEGIVVAVGPGKINENGRHEPVSLEVGARVLFSSYAGTEVKSPKDEETYLILNADDILGVFA

SEQ ID NO: 41 - protein, 129 aa, Parachlamydia acanthamoebae; identical to SEQ ID NO: 38

SEQ ID NO: 42 - protein, 91 aa, Parachlamydia acanthamoebae
GSNWSSHNFFRVLYLILNSLFLKTITEVNMATERTLSIIKPDAVGNNHIGDIIARFEKAGIRIVAAKMKHLSQADAEGFYAVHKERPFSVT

SEQ ID NO: 43 - protein, 164 aa, Parachlamydia acanthamoebae
RNVMYSYLCILLTVISTSLFAHQPNTTEQQTLSIIKPDAVSSKHIGHIISRFEDNGLRVAAVKMTKLSKEQAGQFYAVHKERPFYTDLVEFMSSGPVVILVLKGEDAVAKNRALMGATDPSKAEKNTLRADFAQSVTKNAVHGSDSVENAEKEITFFFNPSEIY

SEQ ID NO: 44 - protein, 193 aa, Parachlamydia acanthamoebae
KFNFLQEAIMTKAVFGLAKNESQAQRIVQKLQASGFDTDEISVLFADRTHEGKLTDVSTNKGAIGHEKHTKAPEGAATGAVSGGIIGGSIGILAGIGALAIPGLGPFIAAGPIMAALAGSGIGGSLGMLIGALVGMGIPEYEAKRYENSLKAGGILISVHTNNAEDVDRASELLRKEGAEDISTSYEKSERSS

SEQ ID NO: 45 - protein, 186 aa, parachlamydia acanthamoebae
KEGIMKNQDLTQLFIDELADMYNSENQILKALPKLIKAASLPDLKEALTTHLHETEEQVERIEKIFSLMNLPVSDKICEAMRGLIKEADDLTQNKSKSATLDAAIISAAQKVEHYEIASYGTLRSFAKHLDLKSEITDLLQETLDEEGSADKKLTKIADGSFLSSGVNKEAVEEEFAHSSSHRKRK

SEQ ID NO: 46 - protein, 217 aa, Parachlamydia acanthamoebae
EGSLFHNFFNPICRCFILHRSASILKVKTIMAEKKNQLFPGREPERPLECTECKKPIAVRYTEIIGNSINNISMCADCPELKKKLYGVQSHKEGGVSVGDGGANLACGNCGTTLEEVRIGSRMGCSTCYDVFGEALLADMVSSNKIPARLASNKKSIPFHIGRTPQEAPIINSSLRLLALNEALNETLQREDYEQAALLRDQIKELTKDAEEGHDEK

SEQ ID NO: 47 - protein, 127 aa, Parachlamydia acanthamoebae
FKKYYNKTYHIPLRRCVMFPRKFLFLSLFALSCSSLAFSTQSTLLARGGEHEGGGHHEGQHHEMNHHENGQRFHDEGHGNEFRHGYGHEGEWNNNRHGVFVDESNPAPVVVEPSDDVPYNSSNGYGN

SEQ ID NO: 48 - protein, 189 aa, Parachlamydia acanthamoebae
VRNGDQLEEKTMRKLLAYGLCLFLLTSCNSSEQKAEDPHHIKKANAVVNPTEGYKTWGNITFTETKEGVQIVANVQGLPAGKHGFHIHEFGDCSAPDASSAGAHYDPTHHKHGGPDDLDRHVGDLGNLEANENGHALYERLDKTITLNGPNSIVGKSIVIHEDEDDFKSQPAGNSGKRVACGLILPLEE

SEQ ID NO: 49 - protein, 219 aa, Parachlamydia acanthamoebae
TLLDAMVIMEKFEKGQDKIQKICDTLRKETLEPARQEAQEIIAEAHAKAAKIIKEAEQQAVTLHAQARKSIEQERNVFQSSLEQAARQGLESLRQSIEHKLFNEELEGLLEKQTADPKLIANVVNAIVEAVQKEGISSNLSAVISKRVSPEQVNALLLENVKSKLNEKGVTLGNFSGGAEVKLHGKRLTIDITDQTLKELLAGYARKDFRQLIFGIKGG

SEQ ID NO: 50 - protein, 277 aa, Parachlamydia acanthamoebae
QMQEQPKRCSEFREGGRRMATAADKIIKKELDKLGSEITKEQRKSYEDVLNKIFKDGQSPQQAMGLSDETLEAFYGYAYNLYGSGNYLDAQRIFQFLINLDASRSKYALGLAASMHMQKNYQAAIEAYMLAAFYDQENPVPFFHIADCYIKLDNPYGALLALDDAIRLGFYPRYTLLRSKAILMRKKIRDDLGLPDTPPPIKEYLILQGCDVEGLDIEKLFLPGSEGEEEKFEEAVKKAEGTSQEREKAQMIVQESSESGDVDLEPGRQWDPPKLEL

SEQ ID NO: 51 - protein, 277 aa, Parachlamydia acanthamoebae
GVEIMDINSQLPSNHSNPSIPFNQPSQSVEFDRLMAQSQSNNLPFLTGILSLENLKKIELGETDHVEIRMTHDLLEFVEAIKNNTAVMIQTSHANTPVFKFSKEINRLIHDQHVHEFTVSLGDQTKGYHRNAVQTEKYVLEDMRALDAHFLEELTLFFQGNHQNSQDSYKEQQKSIASDYQKSQQRLGKMRERALLNQEKEEDHPKIEMEKEKVPLPEKAVGEGTLFDQIVDKQERASIEAQEQKEQTAEVELKKTAKAKNLQIDGDNISMAKREGS

SEQ ID NO: 52 - protein, 584 aa, Parachlamydia acanthamoebae
NIKYVINLYLVVVFFSCHALLASDSLLRQEMVEDLHFIRKNFQIKYVPADWKLLQFGWNLNQQFDNAEAEITSLSSPTLKDYHKILNKLFTSAADYHVNVSFYSTEMASLPFHLQGAEGRYFITWINEEKLPEECLDWSIGDEVIAFDGQDIHEFVQNLRRSEHDRHNEKTDQRFAERSLTLRAGMFAEDVPQGNVEVDLIHKKTGEQKSYVLTWTYHPEQVKEIPLPSLVARGISEKQPFFQRPYYKKMMLTPLYAKFNQPESRVVEDVHNLGARKSFVPPLGSIVWQSPPDSPFHAYICETPSRQRIGYIRIATYDGTDDEVVEFAKLIDYFNPRTQALVVDQVNNPGGSVFYSYGLLSLLTDQPLELPKYKMAITQEEVMEALETLDTLKEITNDEEAIDVCGPSLHGYAVSFELVQSVIQYFQFVLSEWEEGKYVTSPVHLLVNTIAPSPFVTYDKPMVVLVNEFDISCGDSFPCILQDNQRALIFGTRTAGAGGTVLGGGHPNRLGIAGYSYTASLLERHTDGTPIENLGVTPDVSYSLTPEDFQNGYKGYKAALQEAIDGLLKNTRPSSSACPLRDRH

SEQ ID NO: 53 - protein, 628 aa, Parachlamydia acanthamoebae
RLGSNTKPQFNQKLRSNSHQPRANSKCGKGDSENDCKQIEKSTKDVKKGVFIMRKQLGVITSLLFLCATAFLGAAHSSPNNYGWGGGYPVDEQQSYGQGYEQSYAPGPQGSYAPAYEQSSRPRQACPQPACPQPRTCPAPRQACPQPAPVCKPVCVPPQPACCEEPLCKTITPCRHGNQNKLVCHDGITVTARNPKMCMLGDQYPLEFDIQACDDVCNVVVTTHLPEGVSFVRSVPEAKVDGNKLVWEIGSMEKGQCVPAKVWVKCECEGELCACFCATASPVRFCSLLCAKPILTCHKCGPEEVCPGDQVNYTITVTNRGSCAAEDVVVTDNVPEGLEHSSCLRTLCYKLGTLEPCQTKKINLCFTAVKRGQVCNTAVVTACNADTVSCQWCTNVCKECIEITKVGPKEVPIGKNADYQITVTNPGDKDLTEVTITDCAPSSTSIVSANGATIRGNQAVWRLKELKPGEKVTLPITLTTCTPGCWTNRVTVTNCQNCTACAEATTRWRGRPALNMCITDTEDPICIGESTTYCITVTNQGSEADSNVAVVVRFPKEVTPTAAVGDSAGTVSGQTVTFAPVANFAPRQTLKFRIDARAKESGDARIIAEVSSDSIKTPIVQQESTIVN

SEQ ID NO: 54 - protein, 395 aa, Parachlamydia acanthamoebae
PDWNIQQGLSDSRVNVTLEAMKQVQQETKEELTAEQAESKLAFQESQKEISNPFAARLTKKEKPLSAQRGRVQKSGKMGEKADRLLPLQAIKDAASQFQGRNPELKAQILVLLREQIKPDDSKEEILRKILEFYPDVSLADEALEFLLETTDGELYQKIKEIKEEFSQQNDREIAAGRNISAQARQAADAGLGTPTTLRDMYRDITGTPRDSTSLFQELSQRYPFNELKKVVSFLLHSLGSDLKSKGPSIPRGQLHRLITETRSLQAILGVYRFFQGRMNLMHSLFSKEGLDFPEQLTFEMMAKQFMSLAAERYPSADKVLQRAVKLGIEDWILAKIIAFSQFRDAVREVAMNQIYKSLQHRDELLLAIIEALEDLEDDLEEQEEKEQEEEEDKE

SEQ ID NO: 55 - protein, 427 aa, Parachlamydia acanthamoebae
KKWHVILYECALAIAGVCALPWLIYQAIFKKKYRKSFWKRLGWGFPAIQKEQRTVIWMHAVSVGETKAIIGLARLFKERFSNSLLIISSITETGHEEAQRSLPFADHHVYLPFDFGWMIKPIIKRISPDIVILSETDFWFNFLHQAKQSGAFLSVVNGKLSERSLKRYQMLGSFSLFSLFDLFCLQNTQYQNRFSSLNIPLTKLSVTGNLKCGTMLPRLSEQEIVDWREKLGFSTQDLVLTIGSTHDPEERELLEQLQPLLQKFPQLKILLVPRHPERCEQVAQLLHQSDIPYERYSANASKEQARVLLVDAMGVLLKCYQLSDVAIVAGSFIEKVGGHHILEPSYYEVPVLFGPHMHSQREFEALCLEHGAGLQVNYEDIAHSVEMLLNDEALRKSIGKKGFQLMLELQGAHEVTFKTLQQHLKRI

SEQ ID NO: 56 - protein, 375 aa, Parachlamydia acathamoebae
MQRKVILIVFLCLFQIACGSEIDNEELEENAFANEETQNFELSEVEHLQENDASTECCERPYFKLSAELIYLKPSLDQASYVISSSNNIVNGEFFPHGKRHNNTSSYKPGFRIEAQYEPCQSANALDFRFTYLNSSNSDSTSGRFLFDTIGFPGDGAQAPEDINYAGKAHIRDNYRYHSLDATFNRLALDSCLDNLFLLMGLHYAHVGHTTHFKSRGVFPDHSVTKPVNNRLKSHSDFWGIGPQFGLEYTYNLTSPSCCFGRLALNTNLRASILCSQSNASFHYKTLRTAGSKGIKLRNDDLWRVNPAFDAKIGANYTLLLCNFEATVELGYEWLWYHHSVDSITGIDVAFAGDSIDLYSNLSLHGPFLRVNIAF

SEQ ID NO: 57 - protein, 325 aa, parachlamydia acanthamoebae
MTYFMRKIYVAVTLLSITSLGYANEDYDNSLTCGENENDFCCEPISCGLGFISADLLYWRAFESGLDACVPGQVSDIVTSDGKVVSRFKGRGRDPHFDWNPGFRIAAGYELPCSDLDIAASWTYFHSRSHGPRNHGNKIRWNLNFDAVDVVAGYESDFGACFALRPFGGLRGAWIDQKLRIHGTSNSASSSVTNDLLEAKTKNKENFWGVGPLIGLGADWNIGCGFSLYTSASIAWLYGNIDVRLREADETVDTVNLCRVKKRLDANLAVADAALGIRWQTCFCRNTRLILQLGLEHHRYFDYNRIGGYGDLCFDGFNFSAGLEF

SEQ ID NO: 58 - DNA, 30 bp, Artificial
  Primer forward Pac1 P. acanthamoebae
cagcatatgaattttaaatcttcctttaca

SEQ ID NO: 59 - DNA, 34 bp, Artificial
  Primer rv Pac1 P. acanthamoebae
gatgagctcttaatcgactttaatagaaacgaag

SEQ ID NO: 60 - DNA, 33 bp, Artificial
  Primer forward Pac4 P. acanthamoebae
gatcatatggctataagtttatattcaaatcaa

SEQ ID NO: 61 - DNA, 33 bp, Artificial
  Primer Pac4 reverse P. acanthamoebae
gatggatccttaggtattagacgttgcaggttt

SEQ ID NO: 62 - DNA, 34 bp, Artificial
  Primer Pac12 forward P. acanthamoebae
gatcatatgacttacaataataatatcaatgtat

SEQ ID NO: 63 - DNA, 32 bp, Artificial
  Primer Pac12 reverse P. acanthamoebae
gatggatccctacggaatttgatcggaacgtt

SEQ ID NO: 64 - DNA, 33 bp, Artificial
  Primer Pac18 forward P. acanthamoebae
gattcatgagtccagatccaatcaaaggtttcg

SEQ ID NO: 65 - DNA, 32 bp, Artificial
  Primer Pac18 reverse P. acanthamoebae
gatctcgagctgtgtaagtccttggaacatct

SEQ ID NO: 66 - DNA, 31 bp, Artificial
  Primer Pac26 forward P. acanthamoebae
gaacatatgaatattaatagtactccacctg

SEQ ID NO: 67 - DNA, 35 bp, Artificial
  Primer Pac26 reverse P. acanthamoebae
gtcggatccttagtgaatacgataattaaatgcgc

SEQ ID NO: 68 - DNA, 27 bp, Artificial
  Primer Pac35 forward P. acanthamoebae
gatcatatgcaacgatcccttgggggt

SEQ ID NO: 69 - DNA, 31 bp, Artificial
  Primer Pac35 reverse P. acanthamoebae
gatggatccttagtattggctaccgcagcaa

SEQ ID NO: 70 - DNA, 36 bp, Artificial
  Primer Pac123 forward P. acanthamoebae
gactcatgagtaaaaaatggcatgtgattttatacg

SEQ ID NO: 71 - DNA, 32 bp, Artificial
  Primer Pac 123 reverse P. acanthamoebae
gacctcgagaatgcgtttaagatgctgttgca